(19) United States
(12) Patent Application Publication
(10) Pub. No.: US 2022/0324934 A1
(43) Pub. Date: Oct. 13, 2022

(54) DESIGNED IL-2 VARIANTS

(71) Applicant: The Board of Trustees of the Leland Stanford Junior University, Stanford, CA (US)

(72) Inventors: Kenan Christopher Garcia, Menlo Park, CA (US); Possu Huang, Stanford, CA (US)

(21) Appl. No.: 17/741,095

(22) Filed: May 10, 2022

Related U.S. Application Data
(63) Continuation of application No. PCT/US2021/021137, filed on Mar. 5, 2021.
(60) Provisional application No. 62/985,694, filed on Mar. 5, 2020.

(51) Int. Cl. C07K 14/55; A61P 35/00; G16B 15/30; G16B 35/10

(52) U.S. Cl. CPC C07K 14/55; A61P 35/00; G16B 15/30; G16B 35/10; A61K 38/00

(57) ABSTRACT

Variant IL-2 proteins and uses thereof are provided. In some embodiments, the IL-2 variant proteins have greater potency for activation of IL-2 signaling pathways in cells lacking CD25 expression, relative to wild-type IL-2.

### CROSS-REFERENCE TO RELATED APPLICATIONS

This application is a continuation and claims the benefit of PCT Application No. PCT/US2021/021137, filed Mar. 5, 2021, which claims priority to U.S. Provisional Patent Application No. 62/985,694, filed Mar. 5, 2020, which applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on March 5, 2021, is named STAN-1701WO Seq listing_ST25.txt and is 24 kb in size.

### BACKGROUND

Interleukin-2 (IL-2) is a type I α-helical cytokine that functions as a multi-lineage lymphocyte growth factor. On activated lymphocytes and Treg cells, IL-2 signals through a high-affinity (10 pM) heterotrimeric receptor complex, consisting of the IL-2Rα (CD25), IL-2Rβ (CD122), and γc (CD132) chains. In resting lymphocytes it signals via the intermediate-affinity (1 nM) heterodimeric receptor complex, consisting of the IL-2Rβ and γc chains. The IL2-Rα subunit is constitutively expressed in high levels on regulatory T cells (Treg) and at lower levels on natural killer (NK) cells and resting effector CD8+ T cells, resulting in differential IL-2 potency between different immune cell compartments. From a clinical perspective, both IL-2 agonism and antagonism is of considerable importance and it has been used for more than three decades towards immunotherapies of malignancies.

The plasticity of IL-2 has been an important parameter in the generation of small molecules that bind to IL-2 and block signaling; engineering ‘super-agonist’ variant versions of IL-2 with high affinity for IL-2Rβ; and isolation of antibodies that bind to IL-2, resulting in biasing its actions to different immune cell subsets. Indeed, many efforts have partially focused to improve its therapeutic potential by manipulating its ability to selectively target specific cell types. In one approach, monoclonal antibodies against IL-2 can alter its properties by binding to a number of distinct conformational epitopes, thereby modifying the interactions of IL-2 with any of the IL-2R subunits and resulting in the proliferation of either Treg or Teff cells. For example, wild-type mouse IL-2 (mIL-2) can be administered in complex with an anti-mouse IL-2 monoclonal antibody (JES6-1) and used to preferentially induce Treg cell proliferation.

IL-2 can induce the expansion of T cells to enhance adoptive immunotherapy and is approved by the FDA for the treatment of melanoma and renal cell carcinoma, with complete remission in a subset of patients. However, IL-2 can also promote pathologic responses, and a therapeutic goal is to maintain the desired actions of this cytokine while blocking untoward deleterious responses.

We have previously used the structure of the high-affinity IL-2-IL-2R complex to develop IL-2 “superkines” with augmented action due to enhanced binding affinity for IL-2Rβ, which eliminates the functional requirement for IL-2Rα. The super-IL-2 platform was also used to generate variants that retain increased binding affinity for IL-2Rβ but that exhibited decreased binding to γc and thereby have defective IL-2Rβ-γc heterodimerization and signaling.

Continued refinement and development of human IL-2 signaling variants is of great interest for clinical purposes, including the treatment of cancer and autoimmune diseases. Variant proteins for this purpose are provided herein.

### SUMMARY

Variant IL-2 proteins and uses thereof are provided. In some embodiments, the IL-2 variant proteins have greater potency for activation of IL-2 signaling pathways in cells lacking CD25 expression, relative to wild-type IL-2. Such proteins may be referred to herein as IL-2 superagonists. The EC50 for a superagonist on CD25− cells may be reduced at least two-fold relative to the wild-type protein, at least 3-fold relative to the wild-type protein, at least 4-fold relative to the wild-type protein, at least 5-fold relative to the wild-type protein, for example an EC50 of less than about 2 nM, less than about 1.5 nM, less than about 1 nM, less than about 0.5 nM, etc.

In one embodiment, provided herein is an IL-2 superagonist variant having increased binding affinity for CD122 relative to wild-type IL-2, where the binding affinity has a Kd less than about 10-9, less than about 10-10, or less that about 10-12. Such variants are exemplified, for example, by the variants of SEQ ID NO: 1, SEQ ID NO: 7 and SEQ ID NO: 8 or alternatively in revertant sequences in which the residues at positions 69 and 115 are reverted back to wild-type valine residues. Other variants have a greater potency for activation of IL-2 signaling pathways in cells lacking CD25 expression, but do not have a substantially increased binding affinity relative to the wild type IL-2 protein. Such variants are exemplified, for example, by the variants of SEQ ID NO: 6, SEQ ID NO: 15, SEQ ID NO: 16, and SEQ ID NO: 17 or alternatively in revertant sequences in which the residues at positions 69 and 115 are reverted back to wild-type valine residues.

In other embodiments, the variant IL-2 proteins have greater selectivity for CD25 expressing cells, and have reduced potency for activation of IL-2 signaling pathways in cells lacking CD25 expression, relative to wild-type IL-2. Such proteins may be referred to herein as CD25 selective agonists.

IL-2 variants find use, for example, as IL-2 selective agonists and superagonists in applications where modulation of IL-2 signaling is desirable, for example by upregulation of IL-2 signaling for treatment of cancer; and for select activation of IL-2 signaling in autoimmune diseases, and the like. Also provided are nucleic acids encoding such IL-2 variants, methods of making such IL-2 variants, pharmaceutical compositions that include such IL-2 variants and methods of treatment using such IL-2 variants.

In some embodiments, the IL-2 variant comprises one or more amino acid substitutions that increase IL-2 potency in CD25− cells, selected from substitutions at amino acid positions 27, 28, 31, 32, 35, 39, 52, 72, 74, 75, a region comprising residues 74-80, 85, 86, and 92, numbered in accordance with wild-type hIL-2.

In some embodiments the IL-2 variant comprises one or more of the amino acid changes: G27M; I28L; Y31A; K32D/E/S; K35S; M39L or alternatively M39I; E52S or alternatively E52D, E52N, E52T; L72A/Q/D/L/H/T; loop: GDDPKTI, DSTDETV, DSTDERI, DSTDSRI, SKDQDKV, ADDKDTI, ADDQDKI, AQSKNFHL,SKDQKKV, SDDQDKV; L80V/I; R81Δ; P82Δ; L85V or alternatively L85M or L85A; I86V; and I92F.

In one embodiment, the IL-2 variant having a greater binding affinity for CD122 as compared to wild-type human IL-2, comprises the amino acid changes: G27M; I28L; K32D; M39L; E52S; V69A; L72A/Q/D/L/H/T; loop: GDDPKTI; L80I; R81Δ; P82Δ; L85V; I86V; I92F; and V115I, for example as set forth in SEQ ID NO: 1 or alternatively in revertant sequence of SEQ ID NO: 1 in which the residues at positions 69 and 115 are reverted back to wild-type valine residues.

In one embodiment, the IL-2 variant having a greater binding affinity for CD122 as compared to wild-type human IL-2 comprises the amino acid changes: G27M; I28L; K32D; V69A; L72Q; loop: DSTDETV; L80V; R81Δ; P82Δ; and V115I, for example as set forth in SEQ ID NO: 7 or alternatively in revertant sequence of SEQ ID NO: 7 in which the residues at positions 69 and 115 are reverted back to wild-type valine residues.

In one embodiment, the IL-2 variant having a greater binding affinity for CD122 as compared to wild-type human IL-2, comprises the amino acid changes: G27M; I28L; K32D; M39L; E52S; V69A; L72A; loop: DSTDERI; L80I; R81Δ; P82Δ; L85V; I86V; I92F; and V115I, for example as set forth in SEQ ID NO: 8 or alternatively in revertant sequence of SEQ ID NO: 8 in which the residues at positions 69 and 115 are reverted back to wild-type valine residues.

In some embodiments, the IL-2 variant is CD25 selective, and comprises one or more of the amino acid changes: G27M; I28L; K32E; E52S; V69A; L72Q; loop: GDDPKTI; L80I; R81Δ; P82Δ; and V115I, for example as set forth in SEQ ID NO: 6 or alternatively in revertant sequence of SEQ ID NO: 6 in which the residues at positions 69 and 115 are reverted back to wild-type valine residues.

In some embodiments, the IL-2 variant has greater potency on CD25− cells in the absence of greater binding affinity for CD122 and comprises one or more of the amino acid changes: G27M; I28L; Y31A; K32D/E/S; K35S; M39L; E52S; V69A; L72A/Q/D/L/H/T; loop: AQSKNFHL; and V115I, for example as set forth in SEQ ID NO: 15 or alternatively in revertant sequence of SEQ ID NO: 15 in which the residues at positions 69 and 115 are reverted back to wild-type valine residues.

In some embodiments, the IL-2 variant has greater potency on CD25− cells in the absence of greater binding affinity for CD122 and comprises one or more of the amino acid changes: G27M; I28L; K32D; M39L; E52S; V69A; L72Q; loop: AQSKNFHL; and V115I, for example as set forth in SEQ ID NO: 16 or alternatively in revertant sequence of SEQ ID NO: 16 in which the residues at positions 69 and 115 are reverted back to wild-type valine residues.

In some embodiments, the IL-2 variant has greater potency on CD25− cells in the absence of greater binding affinity for CD122 and comprises one or more of the amino acid changes: G27M; I28L; K32D; M39L; E52S; V69A; loop: AQSKNFHL; and V115I, for example as set forth in SEQ ID NO: 17 or alternatively in revertant sequence of SEQ ID NO: 17 in which the residues at positions 69 and 115 are reverted back to wild-type valine residues.

In certain embodiments, the subject IL-2 variant has an enhanced capability to stimulate STAT5 phosphorylation in a CD122+ T cell as compared to wild-type hIL-2. In some embodiments, the T cell is a CD8+ T cell. In some embodiments, the subject IL-2 variant has an enhanced capability to stimulate the pERK1/ERK2 signaling in CD122+ T cell as compared to wild-type hIL-2.

In some embodiments the amino acid substitutions described herein are further combined with amino acid substitutions F42A, E61R, R38D, Y45A, or combinations thereof that introduce mutations that affect CD25 binding, e.g. see Thanos et al. (2006) PNAS 103(42):15422-7 and Vazquez-Lombardi et al. (2017) 8:15373.

In another aspect, provided herein is an IL-2 variant fusion protein comprising any one of the IL-2 variants described herein linked to a human Fc antibody fragment.

In another aspect, provided herein is a pharmaceutical composition comprising any one of the IL-2 variants or the IL-2 variant fusion protein described herein and a pharmaceutically acceptable carrier.

In some embodiments, methods are provided for the use of an IL-2 variant provided herein for the treatment of disease. The superagonist IL-2 proteins induce a superior expansion of cytotoxic T cells, leading to improved responses in vivo with less expansion of T regulatory cells and reduced pulmonary edema. Treatment modalities include, without limitation, treatment of cancer, treatment of immunodeficiency, treatment of infectious disease (particularly chronic or difficult to treat infections), use as a vaccine adjuvant, and the like. In such embodiments, an effective dose of the IL-2 variant is administered to an individual.

In some embodiments, methods are provided for the in silico design of cytokine ligands based on crystallographic structures. The design principles are based on the topology of the ligand-receptor interaction. By preferentially stabilizing a cytokine according to its high resolution crystal structure with the receptor, the cytokine can be turned into superagonists. The method identifies locations where packing can be improved in order to increase binding. Core positions are subjected to redesign and tested in a computer model to identify positions where improvements can be introduced. The resulting sequences are analyzed and compared. The computer model is then used to generate backbone structure ensembles while allowing the entire core to redesign while monitoring the residue changes in the identified regions. A subset of residues that improve packing are visually selected. The identities of the subset are held and the redesign performed iteratively until the sequences converge. On identification of candidate sequences, the coding sequence can be generated and expressed for testing.

In some embodiments, the present disclosure provides a variant human IL-2 protein comprising one or more amino acid changes. In some embodiments, the present disclosure provides a variant human IL-2 protein having greater potency for activation of IL-2 signaling pathways in cells lacking CD25 expression relative to wild-type IL-2, comprising one or more amino acid substitutions relative to wild-type human IL-2.

In some embodiments, the one or more amino acid changes is selected from the group consisting of: an amino acid substitution to methionine at position 27 (G27M), an amino acid substitution at position 28, an amino acid substitution to alanine at position 31 (Y31A), an amino acid substitution at position 32, an amino acid substitution to serine at position 35 (K35S), an amino acid substitution at position 39, an amino acid substitution at position 52, an amino acid deletion at position 82 (P82Δ), and an amino acid substitution to isoleucine at position 115 (V115I), relative to the amino acid sequence of wild-type human IL-2 protein represented by SEQ ID NO: 19.

In some embodiments, the one or more amino acid substitutions relative to wild-type human IL-2 is selected from the group consisting of: an amino acid substitution to methionine at position 27 (G27M), an amino acid substitution at position 28, an amino acid substitution to alanine at position 31 (Y31A), an amino acid substitution at position 32, an amino acid substitution to serine at position 35 (K35S), an amino acid substitution at position 39, an amino acid substitution at position 52, and an amino acid substitution to isoleucine at position 115 (V115I).

In some embodiments, the variant human IL-2 protein of the present disclosure comprises an amino acid deletion at position 81 (R81Δ) or a substitution at position 81.

In some embodiments, the variant human IL-2 protein of the present disclosure comprises one or more amino acid substitutions at positions selected from the group consisting of: 69, 72, a region encompassing amino acid residues 74-80, 85, 86, and 92.

In some embodiments, the variant human IL-2 protein of the present disclosure comprises an amino acid substitution at position 28. In some embodiments, the amino acid substitution at position 28 comprises a substitution to leucine (I28L).

In some embodiments, the variant human IL-2 protein of the present disclosure comprises an amino acid substitution at position 32. In some embodiments, the amino acid substitution at position 32 is selected from the group consisting of: a substitution to aspartic acid (K32D), a substitution to glutamic acid (K32E), and a substitution to serine (K32S).

In some embodiments, the variant human IL-2 protein of the present disclosure comprises an amino acid substitution at position 39. In some embodiments, the amino acid substitution at position 39 is selected from the group consisting of: a substitution to leucine (M39L) and a substitution to isoleucine (M39I).

In some embodiments, the variant human IL-2 protein of the present disclosure comprises an amino acid substitution at position 52. In some embodiments, the amino acid substitution at position 52 is selected from the group consisting of: a substitution to serine (E52S), a substitution to aspartic acid (E52D), a substitution to asparagine (E52N), and a substitution to threonine (E52T).

In some embodiments, the variant human IL-2 protein of the present disclosure comprises an amino acid substitution at position 69. In some embodiments, the amino acid substitution at position 69 comprises a substitution to alanine (V69A).

In some embodiments, the variant human IL-2 protein of the present disclosure comprises an amino acid substitution at position 72. In some embodiments, the amino acid substitution at position 72 is selected from the group consisting of: a substitution to alanine (L72A), a substitution to glutamine (L72Q), a substitution to aspartic acid (L72D), a substitution to histidine (L72H), and a substitution to threonine (L72T).

In some embodiments, the variant human IL-2 protein of the present disclosure comprises an amino acid substitution at position 81. In some embodiments, the amino acid substitution at position 81 comprises a substitution to aspartic acid (R81D).

In some embodiments, the variant human IL-2 protein of the present disclosure comprises an amino acid substitution at position 85. In some embodiments, the amino acid substitution at position 85 is selected from the group consisting of: a substitution to valine (L85V), a substitution to methionine (L85M), and a substitution to alanine (L85A).

In some embodiments, the variant human IL-2 protein of the present disclosure comprises an amino acid substitution at position 86. In some embodiments, the amino acid substitution at position 86 comprises a substitution to valine (I86V).

In some embodiments, the variant human IL-2 protein of the present disclosure comprises an amino acid substitution at position 92. In some embodiments, the amino acid substitution at position 92 comprises a substitution to phenylalanine (I92F).

In some embodiments, the variant human IL-2 protein of the present disclosure comprises an amino acid substitution at the region encompassing amino acid residues 74-80. In some embodiments, the amino acid substitution at the region encompassing amino acid residues 74-80 comprises an amino acid sequence of GDDPKTI, DSTDETV, DSTDERI, DSTDSRI, SKDQDKV, SKDQKKV, SDDQDKV, ADDKDTI, or ADDQDKI.

In some embodiments, the variant human IL-2 protein of the present disclosure comprises amino acid changes G27M, I28L, K32D, M39L, E52S, substitution of the region encompassing amino acid residues 74-80 with GDDPKTI, R81Δ, P82Δ, and V115I.

In some embodiments, the variant human IL-2 protein of the present disclosure comprises amino acid changes, wherein the amino acid changes comprise or consist of G27M, I28L, K32D, M39L, E52S, V69A, L72A, substitution of the region encompassing amino acid residues 74-80 with GDDPKTI, R81Δ, P82Δ, L85V, and V115I.

In some embodiments, the variant human IL-2 protein of the present disclosure comprises amino acid changes, wherein the amino acid changes comprise or consist of G27M, I28L, K32D, M39L, V69A, L72A, substitution of the region encompassing amino acid residues 74-80 with GDDPKTI, R81Δ, P82Δ, and (V115I).

In some embodiments, the variant human IL-2 protein of the present disclosure comprises amino acid changes, wherein the amino acid changes comprise or consist of G27M, I28L, K32D, M39L, E52S, V69A, L72A, substitution of the region encompassing amino acid residues 74-80 with GDDPKTI, R81Δ, P82Δ, and V115I.

In some embodiments, the variant human IL-2 protein of the present disclosure comprises amino acid changes, wherein the amino acid changes comprise or consist of G27M, I28L, K32D, M39L, E52S, V69A, L72Q, substitution of the region encompassing amino acid residues 74-80 with GDDPKTI, R81Δ, P82Δ, and V115I.

In some embodiments, the variant human IL-2 protein of the present disclosure comprises amino acid changes, wherein the amino acid changes comprise or consist of G27M, I28L, K32D, M39L, E52S, V69A, L72D, substitution of the region encompassing amino acid residues 74-80 with GDDPKTI, R81Δ, P82Δ, and V115I.

In some embodiments, the variant human IL-2 protein of the present disclosure comprises amino acid changes, wherein the amino acid changes comprise or consist of G27M, I28L, K32E, E52S, V69A, L72Q, substitution of the region encompassing amino acid residues 74-80 with GDDPKTI, R81Δ, P82Δ, and V115I.

In some embodiments, the variant human IL-2 protein of the present disclosure comprises amino acid changes, wherein the amino acid changes comprise or consist of G27M, I28L, K32D, V69A, L72Q, substitution of the region encompassing amino acid residues 74-80 with DSTDETV, R81Δ, P82Δ, and V115I.

In some embodiments, the variant human IL-2 protein of the present disclosure comprises amino acid changes, wherein the amino acid changes comprise or consist of G27M, I28L, K32D, M39L, E52S, V69A, L72A, substitution of the region encompassing amino acid residues 74-80 with DSTDERI, R81Δ, P82Δ, L85V, I86V, I92F, and V115I.

In some embodiments, the variant human IL-2 protein of the present disclosure comprises amino acid changes, wherein the amino acid changes comprise or consist of G27M, I28L, K32D, M39L, E52S, V69A, L72Q, substitution of the region encompassing amino acid residues 74-80 with DSTDSRI, R81Δ, P82Δ, and V115I.

In some embodiments, the variant human IL-2 protein of the present disclosure comprises amino acid changes, wherein the amino acid changes comprise or consist of G27M, I28L, K32D, M39L, E52S, V69A, substitution of the region encompassing amino acid residues 74-80 with SKDQKKV, R81Δ, P82Δ, L85V, I86V, I92F, and V115I.

In some embodiments, the variant human IL-2 protein of the present disclosure comprises amino acid changes, wherein the amino acid changes comprise or consist of G27M, I28L, K32S, M39L, E52S, V69A, substitution of the region encompassing amino acid residues 74-80 with SDDQDKV, R81Δ, P82Δ, L85V, I86V, I92F, and V115I.

In some embodiments, the variant human IL-2 protein of the present disclosure comprises amino acid changes, wherein the amino acid changes comprise or consist of G27M, I28L, K32D, V69A, L72H, substitution of the region encompassing amino acid residues 74-80 with ADDKDTI, R81Δ, P82Δ, and V115I.

In some embodiments, the variant human IL-2 protein of the present disclosure comprises amino acid changes, wherein the amino acid changes comprise or consist of G27M, I28L, Y31A, K32S, K35S, V69A, L72T, substitution of the region encompassing amino acid residues 74-80 with ADDQDKI, R81Δ, P82Δ, and V115I.

In some embodiments, the variant human IL-2 protein of the present disclosure comprises amino acid changes, wherein the amino acid changes comprise or consist of G27M, I28L, K32D, V69A, R81D, and V115I.

In some embodiments, the variant human IL-2 protein of the present disclosure comprises amino acid changes, wherein the amino acid changes comprise or consist of G27M, I28L, K32D, V69A, L72Q, R81D, and V115I.

In some embodiments, the variant human IL-2 protein of the present disclosure comprises amino acid changes, wherein the amino acid changes comprise or consist of G27M, I28L, K32D, M39L, E52S, V69A, R81D, and V115I.

In some embodiments, the variant human IL-2 protein of the present disclosure comprises amino acid changes, wherein the amino acid changes comprise or consist of G27M, I28L, K32D, M39L, E52S, V69A, L72Q, R81D, and V115I.

In some embodiments, the variant human IL-2 protein of the present disclosure comprises amino acid changes, wherein the amino acid changes consist of G27M, I28L, K32D, L72Q, and R81D.

In some embodiments, the variant human IL-2 protein of the present disclosure has a greater potency for activation of IL-2 signaling pathways in cells lacking CD25 expression. In some embodiments, the variant IL-2 protein has an EC50 for activation of IL-2 signaling pathways in cells lacking CD25 expression at least two-fold lower than the wild-type protein. In some embodiments, the variant IL-2 protein has increased binding affinity for human CD122. In some embodiments, the variant IL-2 protein has a greater potency for activation of IL-2 signaling pathways in cells lacking CD25 expression, but does not have a substantially increased binding affinity relative to the wild type IL-2 protein.

In some embodiments, the variant human IL-2 protein of the present disclosure is fused to a human IgG Fc domain. In some embodiments, the IgG Fc domain is an IgG1, IgG2, IgG3, or IgG4 Fc domain.

In some embodiments, the present disclosure provides a nucleic acid encoding a variant human IL-2 protein of the present disclosure. In some embodiments, the present disclosure provides a vector comprising a nucleic acid, wherein the nucleic acid encodes a variant human IL-2 protein of the present disclosure. In some embodiments, the present disclosure provides a recombinant cell comprising a nucleic acid, wherein the nucleic acid encodes a variant human IL-2 protein of the present disclosure. In some embodiments, the present disclosure provides a recombinant cell comprising a vector disclosed herein.

In some embodiments, the present disclosure provides a pharmaceutical composition comprising a variant human IL-2 protein as disclosed herein, and a pharmaceutically acceptable carrier.

In some embodiments, the present disclosure provides a method of treating disease in a subject in need thereof, the method comprising administering to the patient a variant human IL-2 protein disclosed herein, or a pharmaceutical composition disclosed herein.

In some embodiments, the present disclosure provides a method of treating a disease in need of expansion of T cells, the method comprising administering an effective amount of a variant human IL-2 protein disclosed herein or a pharmaceutical composition disclosed herein. In some embodiments, the disease is a hyperproliferative disease. In some embodiments, the disease is cancer.

In some embodiments, the present disclosure provides a method for in silico design of a cytokine based on crystallographic topology of the ligand receptor interaction, the method comprising: identifying locations where packing can be improved in order to increase binding; redesigning core positions and testing in a computer model to identify positions where improvements can be introduced: generating backbone structure ensembles while allowing the entire core to redesign while monitoring the residue changes in the identified regions; visually selecting a subset of resides that improve packing; holding identities of the subset and iteratively performing the redesign until the sequences converge; generating a coding sequence for the in silico designed cytokine.

### BRIEF DESCRIPTION OF THE DRAWINGS

FIG. 1A illustrates exemplary IL-2 variants expressed as fusion proteins to mouse serum albumin (MSA). FIG. 1B shows a representative gel filtration elution profile of MSA-IL-2 wild-type (WT) fusion protein. FIG. 1C shows a representative gel filtration elution profile of MSA-H9-superagonist IL-2 (super-2) fusion protein. FIG. 1D shows a representative gel filtration elution profile of IL-2 variant protein SEQ ID NO: 3 (variant seq-3). FIG. 1E shows a Coomassie-stained SDS-PAGE gel of peak fractions 12-16 from the gel filtration of MSA-H9-super-2 fusion protein from FIG. 1C and peak fractions 12-16 from the gel filtration of MSA-IL-2 WT from FIG. 1B. FIG. 1F shows a Coomassie-stained SDS PAGE Gel of peak fractions 12-16 from the gel filtration of IL-2 variant protein SEQ ID NO: 3 (variant seq3) from FIG. 1D.

FIGS. 2A-2D illustrate design principle I, showing the core design in 4 sub-sections that can potentially be improved. FIG. 2A illustrates the packing between helix 1 and loop 3 of IL-2. FIG. 2B illustrates the packing between helix 2 and helix 3 of IL-2. FIG. 2C illustrates the packing between helix 1 and helix 2 of IL-2. FIG. 2D illustrates the packing between helix 1 and helix 3.

FIGS. 3A-3B illustrate design principle II, showing rebuilding of loop 3 and stabilizing mutations. FIG. 3A illustrates a shortened and idealized Loop 3 showing two clusters tested experimentally. FIG. 3B illustrates a resample and attempt to improve the native IL-2 loop by design.

FIG. 4 are normalized thermal melting curves of selected in silico designed IL-2 variants monitored by circular dichroism. Abbreviations used: wild-type IL-2 (WT); variant IL-2 protein of SEQ ID NO: 1 (seq1); variant IL-2 protein of SEQ ID NO: 14 (seq14); variant IL-2 protein of SEQ ID NO: 15 (seq15); variant IL-2 protein of SEQ ID NO: 16 (seq16); variant IL-2 protein of SEQ ID NO: 17 (seq17); variant IL-2 protein of SEQ ID NO: 6 (seq6); variant IL-2 protein of SEQ ID NO: 7 (seq7); variant IL-2 protein of SEQ ID NO: 20 (super2).

FIG. 5A illustrates a YT-1 CD25− cell, an IL-2 H9 Superagonist IL-2(Super-2)—MSA fusion protein, and IL-2 WT— MSA fusion protein, and exemplary in silico designed IL-2 variant—MSA fusion proteins. FIG. 5B is a graph showing dose-response curves for STAT5 phosphorylation on CD25− YT-1 cells treated with in silico designed IL-2 variant MSA fusion proteins.

FIG. 6A illustrates a YT-1 CD25+ cell, an IL-2-H9 Superagonist IL-2 (Super-2)—MSA fusion protein, and IL-2 WT-MSA fusion protein, and exemplary in silico designed IL-2 variant—MSA fusion proteins. FIG. 6B is a graph showing dose-response curves for STAT5 phosphorylation on CD25+ YT-1 cells treated with in silico designed IL-2 variant MSA fusion proteins.

FIG. 7A is a graph showing dose-response curves for STAT5 phosphorylation on CD25− YT-1 cells treated with a selection of in silico designed IL-2 variant MSA fusion proteins. FIG. 7B is a graph showing dose-response curves for STAT5 phosphorylation on CD25+ YT-1 cells treated with a selection of in silico designed IL-2 variant MSA fusion proteins

FIG. 8 is a table showing amino acid substitutions in designed IL-2 variants (Super-2: variant IL-2 protein of SEQ ID NO: 20; Seq 1: variant IL-2 protein of SEQ ID NO: 1; Seq 2: variant IL-2 protein of SEQ ID NO: 2; Seq 3: variant IL-2 protein of SEQ ID NO: 3; Seq 4: variant IL-2 protein of SEQ ID NO: 4; Seq 5: variant IL-2 protein of SEQ ID NO: 5; Seq 6: variant IL-2 protein of SEQ ID NO: 6; Seq 7: variant IL-2 protein of SEQ ID NO: 7; Seq 8: variant IL-2 protein of SEQ ID NO: 8; Seq 9: variant IL-2 protein of SEQ ID NO: 9; Seq 10: variant IL-2 protein of SEQ ID NO: 10; Seq 11: variant IL-2 protein of SEQ ID NO: 11; Seq 12: variant IL-2 protein of SEQ ID NO: 12; Seq 13: variant IL-2 protein of SEQ ID NO: 13; Seq 14: variant IL-2 protein of SEQ ID NO: 14; Seq 15: variant IL-2 protein of SEQ ID NO: 15; Seq 16: variant IL-2 protein of SEQ ID NO: 16; Seq 17: variant IL-2 protein of SEQ ID NO: 17) compared to WT IL-2 and corresponding EC50 values for STAT5 phosphorylation on CD25+ and CD25− YT-1 cells.

FIG. 9 shows amino acid sequences of in silico designed IL-2 variants (SEQ ID NO: 1 to SEQ ID NO: 17), wild-type IL-2 (SEQ ID NO:19), and Super-2 (SEQ ID NO: 20).

FIG. 10A shows amino acid sequences of WT IL-2 (SEQ ID NO: 19), Super-2 (SEQ ID NO: 20), and in silico designed IL-2 variants of SEQ ID NO: 15 (Sequence 15) and SEQ ID NO: 16 (Sequence 16). FIG. 10B is a table showing amino acid substitutions in IL-2 variants SEQ ID NO: 15 (Seq 15) and SEQ ID NO: 16 (Seq 16) compared to WT IL-2 and corresponding EC50 values for STAT5 phosphorylation in CD25− YT-1 cells and CD25+ YT-1 cells. FIG. 10C is a table showing amino acid substitutions in Super-2 (SEQ ID NO: 20) compared to WT IL-2 and corresponding EC50 values for STAT5 phosphorylation in CD25− YT-1 cells and CD25+ YT-1 cells.

FIG. 11A is a table showing amino acid sequences of WT IL-2 (SEQ ID NO:19), in silico designed IL-2 variant SEQ ID NO: 15 (Seq 15), and IL-2 variant SEQ ID NO: 15 where A69 and I115 were reverted back to WT valine residues (Seq 15 rev), and corresponding EC50 values for STAT5 phosphorylation in CD25− YT-1 cells. FIG. 11B is a table showing amino acid sequences of WT IL-2 (SEQ ID NO:19), in silico designed IL-2 variant SEQ ID NO: 1 (Seq 1), and IL-2 variant SEQ ID NO: 1 where A69 and I115 were reverted back to WT valine residues (Seq 1 rev), and corresponding EC50 values for STAT5 phosphorylation in CD25− YT-1 cells. FIG. 11C is a graph showing dose-response curves for STAT5 phosphorylation on CD25− YT-1 cells treated with MSA-IL-2 variant SEQ ID NO: 1 (MSA-IL2 seq1), MSA-IL-2 variant SEQ ID NO: 1 where A69 and I115 have been reverted to WT valine residues (MSA-IL-2 seq1 rev), MSA-IL-2 variant SEQ ID NO: 15 (MSA-IL2 seq15), MSA-IL-2 variant SEQ ID NO: 15 where A69 and I115 have been reverted to WT valine residues (MSA-IL2 seq15 rev), and MSA-IL-2 WT.

### DETAILED DESCRIPTION

In order for the present disclosure to be more readily understood, certain terms and phrases are defined below as well as throughout the specification.

### Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russell, Molecular Cloning: A Laboratory Manual 3rd ed., Cold Spring harbor Laboratory Press (Cold Spring Harbor, N.Y. 2001), provide one skilled in the art with a general guide to many terms used in the present disclosure. As appropriate, procedures involving the use of commercially available kits and reagents are generally carried out in accordance with manufacturer defined protocols and/or parameters unless otherwise noted.

As used herein, “IL-2” means wild-type IL-2, whether native or recombinant. Mature human IL-2 occurs as a 133 amino acid sequence (less the signal peptide, consisting of an additional 20 N-terminal amino acids), as described in Fujita, et. al., PNAS USA, 80, 7437-7441 (1983). The amino acid sequence of human IL-2 (SEQ ID NO: 18) is found in Genbank under accession locator NP_000577.2. The amino acid sequence of mature human IL-2 is depicted in SEQ ID NO: 19.

SEQ ID NO: 18
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT
SEQ ID NO: 19
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

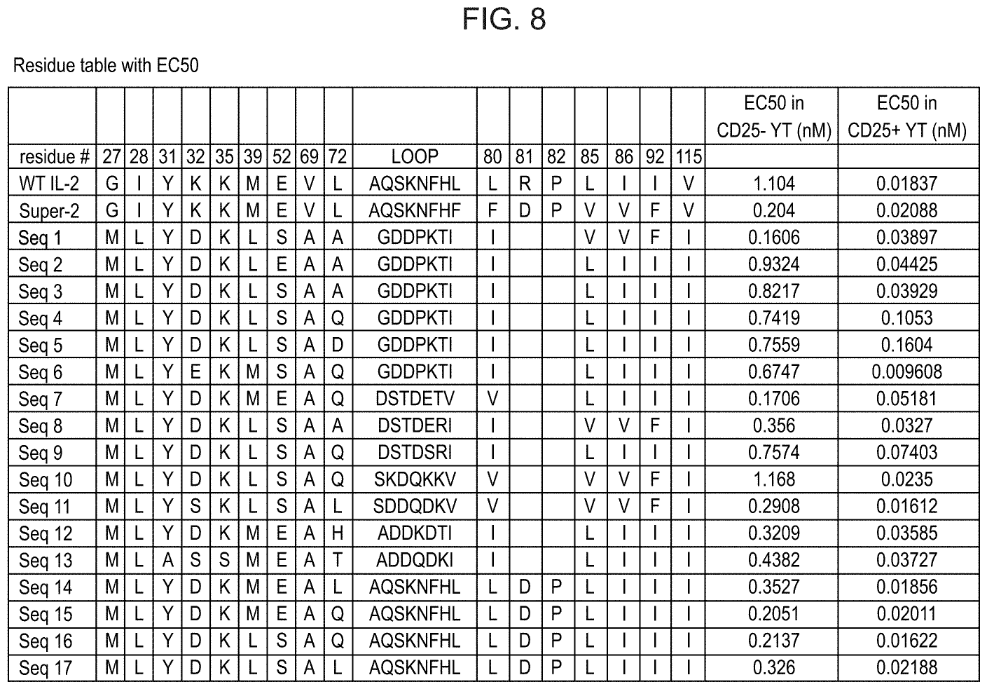

As used herein, “IL-2 variant” means a polypeptide wherein specific substitutions to the interleukin-2 protein have been made. FIG. 8, for example, lists 17 IL-2 variant sequences. Their corresponding relative binding affinities for IL-2Rβ are shown in Table 1. The IL-2 variants can also be characterized by amino acid insertions, deletions, substitutions and modifications at one or more sites in or at the other residues of the native IL-2 polypeptide chain. In accordance with this disclosure any such insertions, deletions, substitutions and modifications result in an IL-2 variant that retains the biological activity of IL-2. Exemplary variants can include substitutions of 1, 2, 3, 4, 5, 6, 7, 8, 9, 10 or more amino acids.

Variants also include conservative modifications and substitutions at other positions of IL-2 (i.e., those that have a minimal effect on the secondary or tertiary structure of the variant). Such conservative substitutions include those described by Dayhoff in The Atlas of Protein Sequence and Structure 5 (1978), and by Argos in EMBO J., 8:779-785 (1989). For example, amino acid substitutions within one of the following groups represent conservative changes: Group I: ala, pro, gly, gln, asn, ser, thr; Group II: cys, ser, tyr, thr; Group III: val, ile, leu, met, ala, phe; Group IV: lys, arg, his; Group V: phe, tyr, trp, his; and Group VI: asp, glu.

“Numbered in accordance with IL-2” means identifying a chosen amino acid with reference to the position at which that amino acid normally occurs in the mature sequence of wild type IL-2, for example G27 refers to the twenty seventh amino acid that occurs in SEQ ID NO: 19.

The term “cell types having the IL-2Rαβγ receptor” means the cells known to have this receptor type, i.e., T cells, activated T cells, B cells, activated monocytes, and activated NK cells. The term “cell types having the IL-2Rβγ receptor” means the cells known to have that receptor type, i.e., B cells, resting monocytes, and resting NK cells.

The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “polypeptide,” “protein” or “peptide” refer to any chain of amino acid residues, regardless of its length or post-translational modification (e.g., glycosylation or phosphorylation). In the event the variant IL-2 polypeptides of the disclosure are “substantially pure,” they can be at least about 60% by weight (dry weight) the polypeptide of interest, for example, a polypeptide containing the variant IL-2 amino acid sequence. For example, the polypeptide can be at least about 75%, about 80%, about 85%, about 90%, about 95% or about 99%, by weight, the polypeptide of interest. Purity can be measured by any appropriate standard method, for example, column chromatography, polyacrylamide gel electrophoresis, or HPLC analysis.

“Operably linked” is intended to mean that the nucleotide sequence of interest (i.e., a sequence encoding an IL-2 variant) is linked to the regulatory sequence(s) in a manner that allows for expression of the nucleotide sequence (e.g., in an in vitro transcription/translation system or in a host cell when the vector is introduced into the host cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression constructs of the invention can be introduced into host cells to thereby produce the human IL-2 variants disclosed herein or to produce biologically active variants thereof.

The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell but are still included within the scope of the term as used herein.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, particle gun, or electroporation.

As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Stemberg disease.

### IL-2 Variants

In various embodiments, the present disclosure provides IL-2 polypeptides, which may be, but are not necessarily, substantially purified and that can function as an agonist of wild-type IL-2R; carrying out one or more of the biological activities of IL-2 (e.g., stimulation of cellular proliferation). IL-2 has been characterized as a T cell growth factor that induces proliferation of antigen-activated T cells and stimulation of NK cells.

An exemplary variant IL-2 polypeptide includes an amino acid sequence that is at least about 80% identical to SEQ ID NO: 19, which may bind the IL-2Rβ with an affinity that is greater than the affinity with which the polypeptide represented by SEQ ID NO: 19 binds the IL-2Rβ. Other IL-2 variants have an enhanced activity in stimulating signaling through CD122 in the absence of increased binding affinity. For example, a variant IL-2 polypeptide can have at least one mutation (e.g., a deletion, addition, or substitution of 1, 2, 3, 4, 5, 6, 7, 8, 9, 10, 11, 12, 13, 14, 15, 16, 17, 18, 19, 20, or more amino acid residues) relative to a wild-type IL-2, and having greater potency for activation of IL-2 signaling pathways in cells lacking CD25 expression, relative to wild-type IL-2. The EC50 for a superagonist on CD25− cells may be reduced at least two-fold relative to the wild-type protein, at least 3-fold relative to the wild-type protein, at least 4-fold relative to the wild-type protein, or at least 5-fold relative to the wild-type protein, for example an EC50 of less than about 2 nM, less than about 1.5 nM, less than about 1 nM, less than about 0.5 nM, less than about 0.3 nM, less than about 0.25 nM, less than about 0.2 nM, less than about 0.15 nM, less than about 0.1 nM, or less than about 0.05 nM.

Exemplary variant IL-2 polypeptides can be at least about 50%, at least about 65%, at least about 70%, at least about 80%, at least about 85%, at least about 87%, at least about 90%, at least about 95%, at least about 97%, at least about 98%, or at least about 99% identical to wild-type IL-2. The mutation can consist of a change in the number or content of amino acid residues. For example, the variant IL-2 can have a greater or a lesser number of amino acid residues than wild-type IL-2. Alternatively, or in addition, an exemplary variant polypeptide can contain a substitution or deletion of one or more amino acid residues that are present in the wild-type IL-2.

By way of illustration, a polypeptide that includes an amino acid sequence that is at least 95% identical to a reference amino acid sequence of SEQ ID NO: 19 is a polypeptide that includes a sequence that is identical to the reference sequence except for the inclusion of up to five alterations of the reference amino acid of SEQ ID NO: 19. For example, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence can occur at the amino (N-) or carboxy (C-) terminal positions of the reference amino acid sequence or anywhere between those terminal positions, interspersed either individually among residues in the reference sequence or in one or more contiguous groups within the reference sequence.

In some embodiments, the present disclosure provides a variant human IL-2 protein comprising one or more amino acid changes. In some embodiments, the amino acid change comprises an amino acid substitution. In some embodiments, the amino acid change comprises an amino acid deletion.

The substituted amino acid residue(s) can be, but are not necessarily, conservative substitutions, which typically include substitutions within the following groups: glycine, alanine; valine, isoleucine, leucine; aspartic acid, glutamic acid; asparagine, glutamine; serine, threonine; lysine, arginine; and phenylalanine, tyrosine. These mutations may be at amino acid residues that contact the IL-2Rβ and/or the IL-2Rγ.

IL-2 variants find use, for example, as IL-2R selective agonists and superagonists in applications where modulation of IL-2 signaling is desirable, for example by upregulation of IL-2 signaling for treatment of cancer; for select activation of IL-2 signaling in autoimmune diseases, and the like. Also provided are nucleic acids encoding such IL-2 variants, methods of making such IL-2 variants, pharmaceutical compositions that include such IL-2 variants and methods of treatment using such IL-2 variants.

In some embodiments, the IL-2 variant comprises one or more amino acid substitutions that increase IL-2 potency in CD25− cells, selected from amino acid positions 27, 28, 31, 32, 35, 39, 52, 69, 72, 74, 75, a region comprising residues 74-80, 85, 86, 92, and 115, numbered in accordance with wild-type hIL-2.

In one embodiment, a minimum set of positions for substitutions that provide enhanced IL-2 potency in CD25 cells may comprise amino acid substitutions at positions 27, 28, 32, 72, and a region comprising residues 74-83. Positions 27, 28 with hydrophobic residues contribute most significantly to structural integrity. Position 72 affects signaling, Q>L. Positions 32 (natively K), 81 (natively R), 52 (natively E) are substituted with consensus helical capping residues, D, N, S or T.

Loop 3 of IL-2 may be wild-type, or may be substituted with a redesigned loop. While the native loop is only 3 residues long, the redesigned structure includes residues preceding and following the helices so that everything between wild-type residue positions 72-84 is rebuilt. The redesigned loop3 sequences share the same structural feature in the 3rd position, for example GDDPKTI. The length is favorable to have a loop 1 or 2 amino acids shorter than the native region from residues 72-83, as shown in the alignments for SEQ ID NO: 1-13.

In some embodiments, the amino acid substitution at position 27 is to G or M. In some embodiments the amino acid substitution at position 28 is to I or L. In some embodiments the amino acid substitution at position 31 is to A, L, M, or Y. In some embodiments the amino acid substitutions at position 39 is to L, I, or M. In some embodiments the amino acid substitution at position 85 is to M, V, A, or L. In some embodiments the amino acid substitution at position 86 is to I or V. In some embodiments the amino acid substitution at position 92 is to F or I.

It was found that reverting to wild-type residues as positions 69 and 115 in exemplary designed polypeptides did not affect activity, and therefore the sequences disclosed herein may have the wild-type sequence at those positions.

In some embodiments, the IL-2 variant comprises one or more amino acid changes selected from the group consisting of: an amino acid substitution to methionine at position 27 (G27M), an amino acid substitution at position 28, an amino acid substitution to alanine at position 31 (Y31A), an amino acid substitution at position 32, an amino acid substitution to serine at position 35 (K35S), an amino acid substitution at position 39, an amino acid substitution at position 52, an amino acid deletion at position 82 (P82Δ), and an amino acid substitution to isoleucine at position 115 (V115I), relative to the amino acid sequence of wild-type human IL-2 protein represented by SEQ ID NO: 19.

In some embodiments, the IL-2 variant comprises one or more amino acid substitutions relative to wild-type human IL-2 selected from the group consisting of: an amino acid substitution to methionine at position 27 (G27M), an amino acid substitution at position 28, an amino acid substitution to alanine at position 31 (Y31A), an amino acid substitution at position 32, an amino acid substitution to serine at position 35 (K35S), an amino acid substitution at position 39, an amino acid substitution at position 52, and an amino acid substitution to isoleucine at position 115 (V115I).

In some embodiments, the IL-2 variant comprises an amino acid deletion at position 81 (R81Δ) or a substitution at position 81.

In some embodiments, the variant human IL-2 protein comprises one or more amino acid substitutions at positions selected from the group consisting of: 69, 72, a region encompassing amino acid residues 74-80, 85, 86, and 92.

In some embodiments, the variant human IL-2 protein comprises an amino acid substitution at position 28, 32, 39, 52, 69, 72, 81, 85, 86, and 92.

In some embodiments, the amino acid substitution at position 28 comprises a substitution to leucine (I28L).

In some embodiments, the amino substitution at position 32 is selected from the group consisting of: a substitution to aspartic acid (K32D), a substitution to glutamic acid (K32E), and a substitution to serine (K32S).

In some embodiments, the amino acid substitution at position 39 is selected from the group consisting of: a substitution to leucine (M39L) and a substitution to isoleucine (M39I).

In some embodiments, the amino acid substitution at position 52 is selected from the group consisting of: a substitution to serine (E52S), a substitution to aspartic acid (E52D), a substitution to asparagine (E52N), and a substitution to threonine (E52T).

In some embodiments, the amino acid substitution at position 69 comprises a substitution to alanine (V69A).

In some embodiments, the amino acid substitution at position 72 is selected from the group consisting of: a substitution to alanine (L72A), a substitution to glutamine (L72Q), a substitution to aspartic acid (L72D), a substitution to histidine (L72H), and a substitution to threonine (L72T).

In some embodiments, the amino acid substitution at position 81 comprises a substitution to aspartic acid (R81D).

In some embodiments, the amino acid substitution at position 85 is selected from the group consisting of: a substitution to valine (L85V), a substitution to methionine (L85M), and a substitution to alanine (L85A).

In some embodiments, the amino acid substitution at position 86 comprises a substitution to valine (I86V).

In some embodiments, the amino acid substitution at position 92 comprises a substitution to phenylalanine (I92F).

In some embodiments, the variant human IL-2 protein comprises a substitution of the region encompassing amino acid residues 74-80. In some embodiments, the amino acid substitution at the region encompassing amino acid residues 74-80 comprises an amino acid sequence of GDDPKTI, DSTDETV, DSTDERI, DSTDSRI, SKDQDKV, SKDQKKV, SDDQDKV, ADDKDTI, or ADDQDKI.

In some embodiments, the variant human IL-2 protein comprises amino acid changes

G27M, I28L, K32D, M39L, E52S, substitution of the region encompassing amino acid residues 74-80 with GDDPKTI, R81Δ, P82Δ, and V115I.

In some embodiments, the variant human IL-2 protein comprises amino acid changes, wherein the amino acid changes comprise or consist of G27M, I28L, K32D, M39L, E52S, V69A, L72A, substitution of the region encompassing amino acid residues 74-80 with GDDPKTI, R81Δ, P82Δ, L85V, and V115I.

In some embodiments, the variant human IL-2 protein comprises amino acid changes, wherein the amino acid changes comprise or consist of G27M, I28L, K32D, M39L, V69A, L72A, substitution of the region encompassing amino acid residues 74-80 with GDDPKTI, R81Δ, P82Δ, and V115I.

In some embodiments, the variant human IL-2 protein comprises amino acid changes, wherein the amino acid changes comprise or consist of G27M, I28L, K32D, M39L, E52S, V69A, L72A, substitution of the region encompassing amino acid residues 74-80 with GDDPKTI, R81Δ, P82Δ, and V115I.

In some embodiments, the variant human IL-2 protein comprises amino acid changes, wherein the amino acid changes comprise or consist of G27M, I28L, K32D, M39L, E52S, V69A, L72Q, substitution of the region encompassing amino acid residues 74-80 with GDDPKTI, R81Δ, P82Δ, and V115I.

In some embodiments, the variant human IL-2 protein comprises amino acid changes, wherein the amino acid changes comprise or consist of G27M, I28L, K32D, M39L, E52S, V69A, L72D, substitution of the region encompassing amino acid residues 74-80 with GDDPKTI, R81Δ, P82Δ, and V115I.

In some embodiments, the variant human IL-2 protein comprises amino acid changes, wherein the amino acid changes comprise or consist of G27M, I28L, K32E, E52S, V69A, L72Q, substitution of the region encompassing amino acid residues 74-80 with GDDPKTI, R81Δ, P82Δ, and V115I.

In some embodiments, the variant human IL-2 protein comprises amino acid changes, wherein the amino acid changes comprise or consist of G27M, I28L, K32D, V69A, L72Q, substitution of the region encompassing amino acid residues 74-80 with DSTDETV, R81Δ, P82Δ, and V115I.

In some embodiments, the variant human IL-2 protein comprises amino acid changes, wherein the amino acid changes comprise or consist of G27M, I28L, K32D, M39L, E52S, V69A, L72A, substitution of the region encompassing amino acid residues 74-80 with DSTDERI, R81Δ, P82Δ, L85V, I86V, I92F, and V115I.

In some embodiments, the variant human IL-2 protein comprises amino acid changes, wherein the amino acid changes comprise or consist of G27M, I28L, K32D, M39L, E52S, V69A, L72Q, substitution of the region encompassing amino acid residues 74-80 with DSTDSRI, R81Δ, P82Δ, and V115I.

In some embodiments, the variant human IL-2 protein comprises amino acid changes, wherein the amino acid changes comprise or consist of G27M, I28L, K32D, M39L, E52S, V69A, substitution of the region encompassing amino acid residues 74-80 with SKDQKKV, R81Δ, P82Δ, L85V, I86V, I92F, and V115I.

In some embodiments, the variant human IL-2 protein comprises amino acid changes, wherein the amino acid changes comprise or consist of G27M, I28L, K32S, M39L, E52S, V69A, substitution of the region encompassing amino acid residues 74-80 with SDDQDKV, R81Δ, P82Δ, L85V, I86V, I92F, and V115I.

In some embodiments, the variant human IL-2 protein comprises amino acid changes, wherein the amino acid changes comprise or consist of G27M, I28L, K32D, V69A, L72H, substitution of the region encompassing amino acid residues 74-80 with ADDKDTI, R81Δ, P82Δ, and V115I.

In some embodiments, the variant human IL-2 protein comprises amino acid changes, wherein the amino acid changes comprise or consist of G27M, I28L, Y31A, K32S, K35S, V69A, L72T, substitution of the region encompassing amino acid residues 74-80 with ADDQDKI, R81Δ, P82Δ, and V115I.

In some embodiments, the variant human IL-2 protein comprises amino acid changes, wherein the amino acid changes comprise or consist of G27M, I28L, K32D, V69A, R81D, and V115I.

In some embodiments, the variant human IL-2 protein comprises amino acid changes, wherein the amino acid changes comprise or consist of G27M, I28L, K32D, V69A, L72Q, R81D, and V115I.

In some embodiments, the variant human IL-2 protein comprises amino acid changes, wherein the amino acid changes comprise or consist of G27M, I28L, K32D, M39L, E52S, V69A, R81D, and V115I.

In some embodiments, the variant human IL-2 protein comprises amino acid changes, wherein the amino acid changes comprise or consist of G27M, I28L, K32D, M39L, E52S, V69A, L72Q, R81D, and V115I.

In some embodiments, the variant human IL-2 protein comprises amino acid changes, wherein the amino acid changes consist of G27M, I28L, K32D, L72Q, and R81D.

In some embodiments, the IL-2 variant comprises one or more of the amino acid changes: G27M; I28L; Y31A; K32D/E/S; K35S; M39L; E52S; V69A; L72A/Q/D/L/H/T; loop: GDDPKTI, DSTDETV, DSTDERI, SKDQDKV, ADDKDTI, ADDQDKI, AQSKNFHL; L80V/I; R81D, L85V; I86V; I92F; and V115I.

In one embodiment, the IL-2 variant having a greater binding affinity for CD122 as compared to wild-type human IL-2, comprises the amino acid changes G27M; I28L; K32D; M39L; E52S; V69A; L72A/Q/D/L/H/T; loop: GDDPKTI; L80I; R81Δ; P82Δ; L85V; I86V; I92F; and V115I, for example as set forth in SEQ ID NO: 1 or alternatively in revertant SEQ ID NO: 1 in which the residues at positions 69 and 115 are reverted back to wild-type valine residues.

In one embodiment, the IL-2 variant having a greater binding affinity for CD122 as compared to wild-type human IL-2, comprises the amino acid changes: G27M; I28L; K32D; V69A; L72Q; loop: DSTDETV; L80V; R81Δ; P82Δ; and V115I, for example as set forth in SEQ ID NO: 7 or alternatively in revertant SEQ ID NO: 7 in which the residues at positions 69 and 115 are reverted back to wild-type valine residues.

In one embodiment, the IL-2 variant having a greater binding affinity for CD122 as compared to wild-type human IL-2, comprises the amino acid changes: G27M; I28L; K32D; M39L; E52S; V69A; L72A; loop: DSTDERI; L80I; R81Δ; P82Δ; L85V; I86V; I92F; and V115I, for example as set forth in SEQ ID NO: 8 or alternatively in revertant SEQ ID NO: 8 in which the residues at positions 69 and 115 are reverted back to wild-type valine residues.

In some embodiments the IL-2 variant comprises one or more of the amino acid changes: G27M; I28L; K32E; E52S; V69A; L72Q; loop: GDDPKTI; L80I; R81Δ; P82Δ; and V115I, for example as set forth in SEQ ID NO: 6 or alternatively in revertant SEQ ID NO: 6 in which the residues at positions 69 and 115 are reverted back to wild-type valine residues.

In some embodiments the IL-2 variant has greater potency on CD25− cells in the absence of greater binding affinity for CD122 comprises one or more of the amino acid changes: G27M; I28L; Y31A; K32D/E/S; K35S; M39L; E52S; V69A; L72A/Q/D/L/H/T; loop: AQSKNFHL; and V115I, for example as set forth in SEQ ID NO: 15 or alternatively in revertant SEQ ID NO: 15 in which the residues at positions 69 and 115 are reverted back to wild-type valine residues.

In some embodiments the IL-2 variant has greater potency on CD25− cells in the absence of greater binding affinity for CD122 comprises one or more of the amino acid changes: G27M; I28L; K32D; M39L; E52S; V69A; L72Q; loop: AQSKNFHL; and V115I, for example as set forth in SEQ ID NO: 16 or alternatively in revertant SEQ ID NO: 16 in which the residues at positions 69 and 115 are reverted back to wild-type valine residues.

In some embodiments, the IL-2 variant has greater potency on CD25− cells in the absence of greater binding affinity for CD122 comprises one or more of the amino acid changes: G27M; I28L; K32D; M39L; E52S; V69A; loop: AQSKNFHL; and V115I, for example as set forth in SEQ ID NO: 17 or alternatively in revertant SEQ ID NO: 17 in which the residues at positions 69 and 115 are reverted back to wild-type valine residues.

An “agonist” is a compound that interacts with a target to cause or promote an increase in the activation of the target.

A “partial agonist” is a compound that interacts with the same target as an agonist but does not produce as great a magnitude of a biochemical and/or physiological effect as the agonist, even by increasing the dosage of the partial agonist.

A “super agonist,” “super-agonist,” or “superagonist” is a type of agonist that is capable of producing a maximal response greater than the endogenous agonist for the target receptor, and thus has an efficacy of more than 100%.

An “antagonist” is a compound that opposes the actions of an agonist, e.g., by preventing, reducing, inhibiting, or neutralizing the activity of an agonist. An “antagonist” can also prevent, inhibit, or reduce constitutive activity of a target, e.g., a target receptor, even where there is no identified agonist.

In certain embodiments, the IL-2 variant has enhanced capabilities to stimulate one or more signaling pathways that are dependent on IL-2Rβ/IL-2Rγc heterodimerization. In some embodiments, the subject IL-2 variant has an enhanced capability to stimulate STAT5 phosphorylation in an IL-2Rβ+ cell as compared to wild-type hIL-2. In some embodiments, the IL-2 variant stimulates STAT5 phosphorylation in an IL-2Rβ+ cell at a level that is 10%, 15%, 20%, 25%, 30%, 35%, 40%, 45%, 50%, 55%, 60%, 65%, 70%, 75%, 80%, 85%, 90%, 95% or more of the level that wild-type IL-2 stimulates STAT5 phosphorylation in the same cell, and may be increased 1-fold, 2-fold, 3-fold, 4-fold or more. In some embodiments, the IL-2 variant stimulates STAT5 phosphorylation in an IL-2Rβ cell at a level that is about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% more than the level that wild-type IL-2 stimulates STAT5 phosphorylation in the same cell. In some embodiments, STAT5 phosphorylation in an IL-2Rβ+ cell is increased about 1-fold, about 2-fold, about 3-fold, about 4-fold, about 5-fold, about 10-fold, about 20-fold, about 50-fold, about 100-fold. In some embodiments, the IL-2R+ cell is a T cell. In particular embodiments, the T cell is a CD8+ T cell. In some embodiments, the CD8+ T cell is a freshly isolated CD8+ T cell. In other embodiments, the CD8+ T cell T cell is an activated CD8+ T cell. In other embodiments, the IL-2Rβ+ cell is a natural killer (NK) cell.

In some embodiments, the variant has an enhanced capability to stimulate ERK1/ERK2 signaling in an IL-2Rβ+ cell as compared to wild-type hIL-2. In some embodiments, the IL-2 variant stimulates pERK1/ERK2 signaling in an IL-2Rβ+ cell at a level that is 10%, 15%, 20%, 25%, 30%, 35%, 40%, 45%, 50%, 55%, 60%, 65%, 70%, 75%, 80%, 85%, 90%, 95% or more of the level that wild-type IL-2 stimulates pERK1/ERK2 signaling in the same cell, and may be increased 1-fold, 2-fold, 3-fold, 4-fold or more. In some embodiments, the IL-2 variant stimulates pERK1/ERK2 signaling in an IL-2Rβ+ cell at a level that is about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% more of the level that wild-type IL-2 stimulates pERK1/ERK2 signaling in the same cell. In some embodiments, the IL-2 variant stimulates and increases pERK1/ERK2 signaling about 1-fold, about 2-fold, about 3-fold, about 4-fold, about 5-fold, about 10-fold, about 20-fold, about 50-fold, about 100-fold. In some embodiments, the IL-2Rβ+ cell is a T cell. In particular embodiments, the T cell is a CD8+ T cell. In some embodiments, the CD8+ T cell is a freshly isolated CD8+ T cell. In other embodiments, the CD8+ T cell is an activated CD8+ T cell. In other embodiments, the IL-2Rβ+ cell is a natural killer (NK) cell.

STAT5 and ERK/2 signaling can be measured, for example, by phosphorylation of STAT5 and ERK1/2 using any suitable method known in the art. For example, STAT5 and ERK1/2 phosphorylation can be measured using antibodies specific for the phosphorylated version of these molecules in combination with flow cytometry analysis as described herein.

In certain embodiments, the IL-2 variant has an enhanced capability to induce lymphocyte proliferation as compared to wild-type IL-2. In some embodiments, the lymphocyte is a T cell. In particular embodiments, the lymphocyte is a primary CD8+ T cell. In other embodiments, the lymphocyte is an activated CD8+ T cell. Cell proliferation can be measured using any suitable method known in the art. For example, lymphocyte proliferation can be measured using a carboxyfluorescein diacetate succinimidyl diester (CFSE) dilution assay or by [3H]-thymidine incorporation, as described herein. In some embodiments, the IL-2 variant induces lymphocyte proliferation at a level that is 1%, 5%, 10%, 15%, 20%, 25%, 30%, 35%, 40%, 45%, 50%, 55%, 60%, 65%, 70%, 75%, 80%, 85%, 90%, 95% or more of the level that wild-type IL-2 induces lymphocyte proliferation. In some embodiments, the IL-2 variant induces lymphocyte proliferation at a level that is about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% more of the level that wild-type IL-2 induces lymphocyte proliferation.

### Recombinant Expression of IL-2 Variants, Expression Vectors and Host Cells

In various embodiments, polypeptides used in the practice of the instant invention are synthetic, or are produced by expression of a recombinant nucleic acid molecule. In the event the polypeptide is a chimera (e.g., a fusion protein containing at least a variant IL-2 polypeptide and a heterologous polypeptide), it can be encoded by a hybrid nucleic acid molecule containing one sequence that encodes all or part of the variant IL-2, and a second sequence that encodes all or part of the heterologous polypeptide. For example, subject IL-2 variants described herein may be fused to a hexa-histidine tag to facilitate purification of bacterially expressed protein, or to a hemagglutinin tag to facilitate purification of protein expressed in eukaryotic cells.

Methods for constructing a DNA sequence encoding the IL-2 variants and expressing those sequences in a suitably transformed host include, but are not limited to, using a PCR-assisted mutagenesis technique. Mutations that consist of deletions or additions of amino acid residues to an IL-2 polypeptide can also be made with standard recombinant techniques. In the event of a deletion or addition, the nucleic acid molecule encoding IL-2 is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap with one another, but blunt-ended fragments can also be ligated. PCR-generated nucleic acids can also be used to generate various variant sequences.

The complete amino acid sequence can be used to construct a back-translated gene. A DNA oligomer containing a nucleotide sequence coding for IL-2 variant can be synthesized. For example, several small oligonucleotides coding for portions of the desired polypeptide can be synthesized and then ligated. The individual oligonucleotides typically contain 5′ or 3′ overhangs for complementary assembly.

In addition to generating variant polypeptides via expression of nucleic acid molecules that have been altered by recombinant molecular biological techniques, subject IL-2 variants can be chemically synthesized. Chemically synthesized polypeptides are routinely generated by those of skill in the art.

Once assembled (by synthesis, site-directed mutagenesis or another method), the DNA sequences encoding an IL-2 variant will be inserted into an expression vector and operatively linked to an expression control sequence appropriate for expression of the IL-2 variant in the desired transformed host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

The DNA sequence encoding the IL-2 variant, whether prepared by site directed mutagenesis, chemical synthesis or other methods, can also include DNA sequences that encode a signal sequence. Such signal sequence, if present, should be one recognized by the cell chosen for expression of the IL-2 variant. It can be prokaryotic, eukaryotic or a combination of the two. It can also be the signal sequence of native IL-2. The inclusion of a signal sequence depends on whether it is desired to secrete the IL-2 variant from the recombinant cells in which it is made. If the chosen cells are prokaryotic, it generally is preferred that the DNA sequence not encode a signal sequence. If the chosen cells are eukaryotic, it generally is preferred that a signal sequence be encoded and most preferably that the wild-type IL-2 signal sequence be used.

### IL-2 Variant Fusion Proteins

As noted above, exemplary subject IL-2 variants can be prepared as fusion or chimeric polypeptides that include a subject IL-2 variants and a heterologous polypeptide (i.e., a polypeptide that is not IL-2 or a variant thereof) (see, e.g., U.S. Pat. No. 6,451,308). Exemplary heterologous polypeptides can increase the circulating half-life of the chimeric polypeptide in vivo, and may, therefore, further enhance the properties of the variant IL-2 polypeptides. In various embodiments, the polypeptide that increases the circulating half-life may be a serum albumin, such as human serum albumin (HAS) or mouse serum albumin (MSA), or the Fc region of the IgG subclass of antibodies that lacks the IgG heavy chain variable region. Exemplary Fc regions can include a mutation that inhibits complement fixation and Fc receptor binding, or it may be lytic, i.e., able to bind complement or to lyse cells via another mechanism, such as antibody-dependent complement lysis.

The “Fc region” can be a naturally occurring or synthetic polypeptide that is homologous to the IgG C-terminal domain produced by digestion of IgG with papain. IgG Fc has a molecular weight of approximately 50 kDa. The variant IL-2 polypeptides can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases. In certain embodiments, the IL-2 variant fusion protein (e.g., an IL-2 partial agonist or antagonist as described herein) includes an IgG1, IgG2, IgG3, or IgG4 Fc region.

In some embodiments, the variant human IL-2 protein is fused to a human IgG Fc domain.

In some embodiments, the IgG Fc domain is an IgG1, IgG2, IgG3, or IgG4 Fc domain.

The Fc region can be “lytic” or “non-lytic,” but is typically non-lytic. A non-lytic Fc region typically lacks a high affinity Fc receptor binding site and a C1q binding site. The high affinity Fc receptor binding site of murine IgG Fc includes the Leu residue at position 235 of IgG Fc. Thus, the Fc receptor binding site can be destroyed by mutating or deleting Leu 235. For example, substitution of Glu for Leu 235 inhibits the ability of the Fc region to bind the high affinity Fc receptor. The murine C′1q binding site can be functionally destroyed by mutating or deleting the Glu 318, Lys 320, and Lys 322 residues of IgG. For example, substitution of Ala residues for Glu 318, Lys 320, and Lys 322 renders IgG Fc unable to direct antibody-dependent complement lysis. In contrast, a lytic IgG Fc region has a high affinity Fc receptor binding site and a C′1q binding site. The high affinity Fc receptor binding site includes the Leu residue at position 235 of IgG Fc, and the C′1q binding site includes the Glu 318, Lys 320, and Lys 322 residues of IgG1. Lytic IgG Fc has wild-type residues or conservative amino acid substitutions at these sites. Lytic IgG Fc can target cells for antibody dependent cellular cytotoxicity or complement directed cytolysis (CDC). Appropriate mutations for human IgG are also known.

In other embodiments, the chimeric polypeptide can include a subject IL-2 variant and a polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag.

In other embodiments, the chimeric polypeptide includes the variant IL-2 polypeptide and a heterologous polypeptide that functions to enhance expression or direct cellular localization of the variant IL-2 polypeptide, such as the Aga2p agglutinin subunit (see, e.g., Boder and Wittrup, Nature Biotechnol. 15:553-7, 1997).

In other embodiments, a chimeric polypeptide including a variant IL-2 and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

### Nucleic Acid Molecules Encoding Variant IL-2

In some embodiments the subject IL-2 variant, either alone or as a part of a chimeric polypeptide, such as those described above, can be obtained by expression of a nucleic acid molecule. Just as IL-2 variants can be described in terms of their identity with wild-type IL-2 polypeptides, the nucleic acid molecules encoding them will necessarily have a certain identity with those that encode wild-type IL-2. In some embodiments, the nucleic acid molecule encoding a subject IL-2 variant can be at least 50%, at least 65%, at least 75%, at least 85%, or at least 95%, at least 99% identical to the nucleic acid encoding wild-type IL-2 (e.g., SEQ ID NO: 19).

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription. In some embodiments, the present disclosure provides a nucleic acid encoding a variant human IL-2 protein of the present disclosure.

Exemplary isolated nucleic acid molecules of the present disclosure can include fragments not found as such in the natural state. Thus, this disclosure encompasses recombinant molecules, such as those in which a nucleic acid sequence (for example, a sequence encoding a variant IL-2) is incorporated into a vector (e.g., a plasmid or viral vector) or into the genome of a heterologous cell (or the genome of a homologous cell, at a position other than the natural chromosomal location).

As described above, the subject IL-2 variant may exist as a part of a chimeric polypeptide. In addition to, or in place of, the heterologous polypeptides described above, a subject nucleic acid molecule can contain sequences encoding a “marker” or “reporter.” Examples of marker or reporter genes include f-lactamase, chloramphenicol acetyltransferase (CAT), adenosine deaminase (ADA), aminoglycoside phosphotransferase (neo.sup.r, G418.sup.r), dihydrofolate reductase (DHFR), hygromycin-B-hosphotransferase (HPH), thymidine kinase (TK), lacz (encoding.beta.-galactosidase), and xanthine guanine phosphoribosyltransferase (XGPRT). One of skill in the art will be aware of additional useful reagents, for example, of additional sequences that can serve the function of a marker or reporter.

The subject nucleic acid molecules can be obtained by introducing a mutation into IL-2-encoding DNA obtained from any biological cell, such as the cell of a mammal. Thus, the subject nucleic acids (and the polypeptides they encode) can be those of a mouse, rat, guinea pig, cow, sheep, horse, pig, rabbit, monkey, baboon, dog, or cat. In one embodiment, the nucleic acid molecules will be those of a human.

### Expression of Variant IL-2 Gene Products

The nucleic acid molecules described above can be contained within a vector that is capable of directing their expression in, for example, a cell that has been transduced with the vector. Accordingly, in addition to the subject IL-2 variants, expression vectors containing a nucleic acid molecule encoding a subject IL-2 variant and cells transfected with these vectors are among the preferred embodiments.

It should of course be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the IL-2 variants to be amplified in copy number.

In some embodiments, the human IL-2 variants of the present disclosure will be expressed from vectors, preferably expression vectors. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are included also.

Exemplary recombinant expression vectors can include one or more regulatory sequences, selected on the basis of the host cells to be used for expression, operably linked to the nucleic acid sequence to be expressed.

The expression constructs or vectors can be designed for expression of an IL-2 variant or variant thereof in prokaryotic or eukaryotic host cells.

Vector DNA can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

In some embodiments, the present disclosure provides a vector comprising a nucleic acid, wherein the nucleic acid encodes a variant human IL-2 protein disclosed herein.

In some embodiments the recombinant IL-2 variants or biologically active variants thereof can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerevisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

The sequences encoding the human IL-2 variants of the present disclosure can be optimized for expression in the host cell of interest. The G-C content of the sequence can be adjusted to levels average for a given cellular host, as calculated by reference to known genes expressed in the host cell. Methods for codon optimization are well known in the art. Codons within the IL-2 variant coding sequence can be optimized to enhance expression in the host cell, such that about 1%, about 5%, about 100/, about 25%, about 50%, about 75%, or up to 100% of the codons within the coding sequence have been optimized for expression in a particular host cell.

In some embodiments, nucleic acid inserts, which encode the subject IL-2 variants in such vectors, can be operably linked to a promoter, which is selected based on, for example, the cell type in which expression is sought. In selecting an expression control sequence, a variety of factors should also be considered. These include, for example, the relative strength of the sequence, its controllability, and its compatibility with the actual DNA sequence encoding the subject IL-2 variant, particularly as regards potential secondary structures. Hosts should be selected by consideration of their compatibility with the chosen vector, the toxicity of the product coded for by the DNA sequences of this invention, their secretion characteristics, their ability to fold the polypeptides correctly, their fermentation or culture requirements, and the ease of purification of the products coded for by the DNA sequences.

In addition to sequences that facilitate transcription of the inserted nucleic acid molecule, vectors can contain origins of replication, and other genes that encode a selectable marker. For example, the neomycin-resistance (neor) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a subject IL-2 variant disclosed herein are also features of the invention. A cell of the invention is a transfected cell, i.e., a cell into which a nucleic acid molecule, for example a nucleic acid molecule encoding a variant IL-2 polypeptide, has been introduced by means of recombinant DNA techniques. The progeny of such a cell are also considered within the scope of the invention.

The expressed polypeptides can be purified from the expression system using routine biochemical procedures, and can be used, e.g., as therapeutic agents, as described herein.

In some embodiments, IL-2 variants obtained will be glycosylated or unglycosylated depending on the host organism used to produce the variant. If bacteria are chosen as the host then the IL-2 variant produced will be unglycosylated. Eukaryotic cells, on the other hand, will glycosylate the IL-2 variants, although perhaps not in the same way as native-IL-2 is glycosylated. The IL-2 variant produced by the transformed host can be purified according to any suitable method. Various methods are known for purifying IL-2. See, e.g. Current Protocols in Protein Science, Vol 2. Eds: John E. Coligan, Ben M. Dunn, Hidde L. Ploehg, David W. Speicher, Paul T. Wingfield, Unit 6.5 (Copyright 1997, John Wiley and Sons, Inc. IL-2 variants can be isolated from inclusion bodies generated in E. coli, or from conditioned medium from either mammalian or yeast cultures producing a given variant using cation exchange, gel filtration, and or reverse phase liquid chromatography.

The biological activity of the IL-2 variants can be assayed by any suitable method known in the art. Such assays include PHA-blast proliferation and NK cell proliferation.

### Methods of Treatment

In some embodiments, variant IL-2 polypeptides, and/or nucleic acids expressing them, are administered to a subject to treat a proliferative or infectious disorder, such as cancer, chronic pathogenic infections, AIDS, etc. In the treatment of such diseases, the disclosed IL-2 variants may possess advantageous properties, such as increased efficacy, decreased toxicity, unexpected additive effects, and unexpected synergistic effects wherein the combination of the variant and the antibody produce more than a simple additive effect. Furthermore, in specific embodiments wherein IL-2 variant treatment has been ineffective at treating abnormal apoptosis and proliferative/differentiative disorders, for example, the combination of the IL-2 variant and the anti-IL-2 antibody is effective.

Examples of disorders include cancer (e.g., carcinoma, sarcoma, metastatic disorders or hematopoietic neoplastic disorders, e.g., leukemias). A metastatic tumor can arise from a multitude of primary tumor types, including but not limited to carcinomas, hematopoietic malignancies, etc. as described above. The compositions of the present invention (e.g., variant IL-2 polypeptides and/or the nucleic acid molecules that encode them) can also be administered to a patient who has a viral infection (e.g., AIDS or an influenza).

Other examples of proliferative disorders include skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer. Examples of skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

Patients amenable to treatment may also have psoriasis. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, psoriasis vulgaris, eruptive (gluttate) psoriasis, psoriatic erythroderma, generalized pustular psoriasis (Von Zumbusch), annular pustular psoriasis, and localized pustular psoriasis.

In some embodiments, the present disclosure provides a method of treating disease in a subject in need thereof, the method comprising administering to the patient a variant human IL-2 protein disclosed herein.

Alternatively, or in addition to methods of direct administration to patients, in some embodiments, variant IL-2 polypeptides can be used in ex vivo methods. For example, cells (e.g., peripheral blood lymphocytes or purified populations of lymphocytes isolated from a patient and placed or maintained in culture) can be cultured in vitro in culture medium and the contacting step can be affected by adding the IL-2 variant to the culture medium. The culture step can include further steps in which the cells are stimulated or treated with other agents, e.g., to stimulate proliferation, or to expand a population of cells that is reactive to an antigen of interest (e.g., a cancer antigen or a viral antigen). The cells are then administered to the patient after they have been treated.

In some embodiments, the present disclosure provides a method of treating a disease in need of expansion of T cells, the method comprising administering an effective amount of a variant human IL-2 protein disclosed herein.

### Pharmaceutical Compositions and Methods of Administration

In some embodiments, variant IL-2 polypeptides and nucleic acids can be incorporated into compositions, including pharmaceutical compositions. Such compositions typically include the polypeptide or nucleic acid molecule and a pharmaceutically acceptable carrier.

A pharmaceutical composition is formulated to be compatible with its intended route of administration. In one embodiment, the variant IL-2 polypeptides are administered orally. In one embodiment, the variant IL-2 polypeptides are administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile- filtered solution thereof.

Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In certain embodiments wherein the administration by inhalation, the variant IL-2 polypeptides, or the nucleic acids encoding them may be delivered in the form of an aerosol spray from pressured container or dispenser which contains a suitable propellant, e.g., a gas such as carbon dioxide, or a nebulizer. Such methods include those described in U.S. Pat. No. 6,468,798. In other embodiments, inhalation is performed using pressurized, non-aerosol administration.

Systemic administration of the variant IL-2 polypeptides or nucleic acids can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the present disclosure provides a method of treating disease in a subject in need thereof, the method comprising administering to the patient a pharmaceutical composition comprising a variant human IL-2 protein disclosed herein.

In some embodiments, the present disclosure provides a method of treating a disease in need of expansion of T cells, the method comprising administering an effective amount of a pharmaceutical composition comprising a variant human IL-2 protein disclosed herein.

In some embodiments, compounds (variant IL-2 polypeptides or nucleic acids) can also be prepared in the form of suppositories (e.g., with conventional suppository bases such as cocoa butter and other glycerides) or retention enemas for rectal delivery.

In some embodiments, compounds (variant IL-2 polypeptides or nucleic acids) can also be administered by transfection or infection using methods known in the art, including but not limited to the methods described in McCaffrey et al. (Nature 41 8:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53: 325, 1996).

In one embodiment, the variant IL-2 polypeptides or nucleic acids are prepared with carriers that will protect the variant IL-2 polypeptide against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

Dosage, toxicity and therapeutic efficacy of such variant IL-2 polypeptides or nucleic acids can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of variant IL-2 polypeptides (e.g., an effective dosage) depends on the polypeptide selected. For instance, single dose amounts of the variant IL-2 polypeptide are in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU) as established by the World Health Organization International Standard for Interleukin-2 (human)). The dosage may be similar to, but is expected to be less than, that prescribed for PROLEUKIN®. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the variant IL-2 polypeptides of the invention can include a single treatment or, can include a series of treatments. In one embodiment, the compositions are administered every 8 hours for five days, followed by a rest period of 2 to 14 days, e.g., 9 days, followed by an additional five days of administration every 8 hours.

The pharmaceutical compositions can be included in a container, pack, or dispenser together with instructions for administration.

The following examples are provided to describe certain embodiments of the invention provided herein and are not to be construed to as limiting.

### EXAMPLES

### EXAMPLE 1: Design, Expression and Purification of IL-2 Variant Proteins

The sequence variants were designed and tested with the goal of making make a better version of IL-2 with a high affinity for IL-2Rbeta or high potency in CD25 negative cells. Computational predicted and designed variants were used to test IL-2 signaling in YT-1 (CD25−) and YT-1 (CD25+) cells. The IL-2 variants, including a wild-type and H9 super-2 control sequence, were cloned into Expi293 expression vector, and transiently expressed from Expi293 cells. IL-2 sequence variants were expressed as MSA fusion proteins and purified by SEC. The effects of the proteins on pSTAT5 signaling was tested in in YT-1 (CD25−) and YT-1 (CD25+) cells.

The design principles are based on the topology associated with IL-2, which suggests intrinsic flexibility in the molecule, based on the observation that the connections between the helices are non-trivial loops with a disulfide linkage, as well as non-idealities in the structural elements: loop arrangements, packing geometry and helical capping. As the dynamics of the structure may be associated with biological function, by preferentially stabilizing IL-2 according to its high resolution crystal structure with the receptor, it was theorized that wildtype IL-2 could be turned into superagonists. Four regions highlighted are locations where design efforts were made to improve the packing.

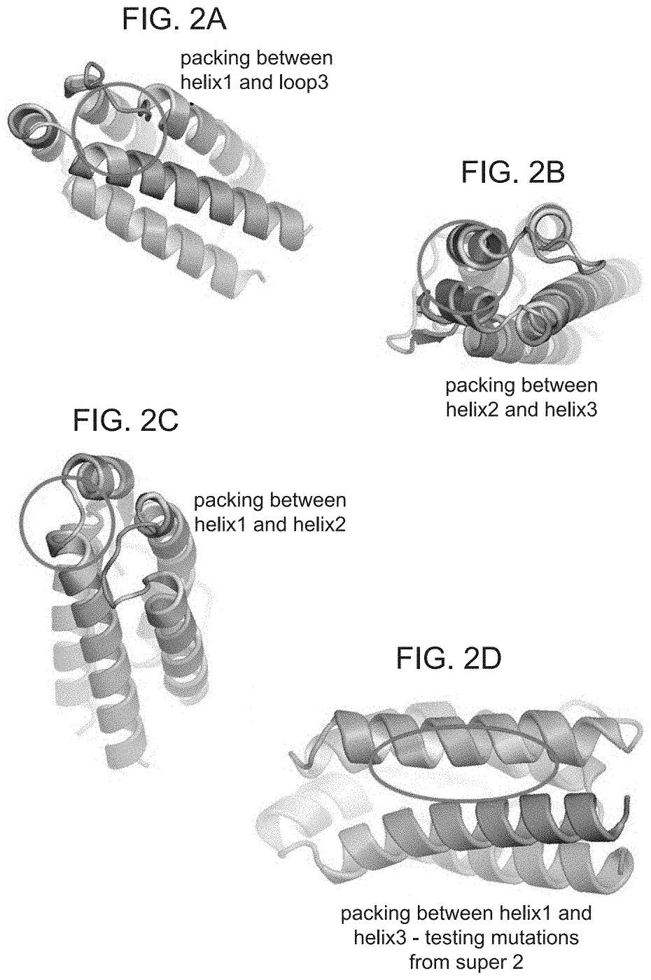

RosettaRemodel, a module in the Rosetta modeling software suite, was used to test the packing quality of the native structure. Core positions are subjected to redesign using normal Rosetta forcefield (Ref2015) and also under softened repulsion parameter settings (soft_rep_design) to identify positions where potential improvements can be introduced. The resulting sequences from running under the two different settings were analyzed and compared. Visual inspection grouped the changes to the different regions highlighted in FIGS. 2A, 2B, 2C, and 2D.

The standard Rosetta forcefield was then used to generate backbone structure ensembles while allowing the entire core to redesign while monitoring the residue changes in the 4 regions. A subset of resides that improve packing were visually selected. We then held the identities of the subset and performed step 3 iteratively until the sequences converge. The 3rd loop was rebuilt, as it is largely disordered in the wild type apoprotein structure and may be responsible for the binding improvements of the evolved super-2 molecule. These steps were repeated for the loop variants.

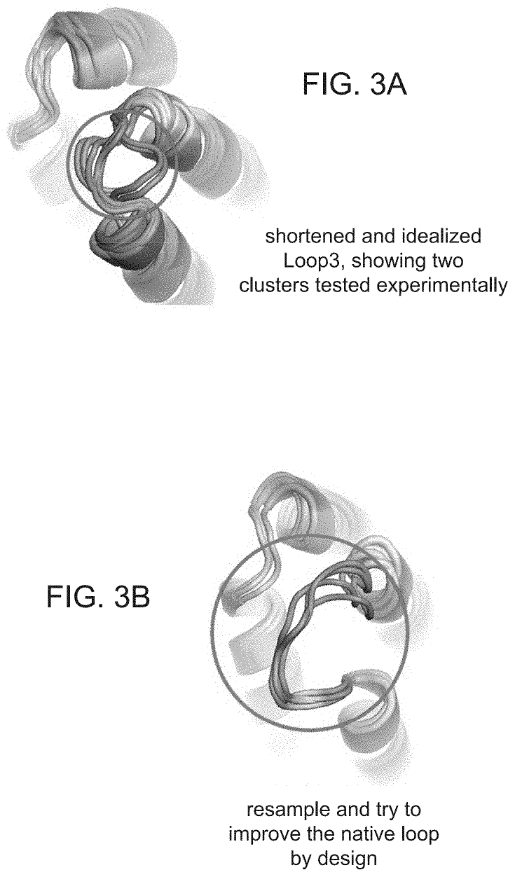

The 3rd loop in the wildtype IL-2 crystal structure is partially disordered. We tried to stabilize it by redesigning new sequences for the original length and, alternatively, to idealize the connection by shortening and rebuilding completely the loop without disrupting the positions of the helices it bridges. As seen in FIGS. 3A and 3B, loop 3 also serves as a conduit between different regions of the core we try to optimize. Rebuilding loop 3 should have the most significant change to the intrinsic structural dynamics of IL-2. The thermodynamics of the overall stability is likely dictated by the packing between the helices. Loop modification mainly should change the folding kinetics and structural dynamics.

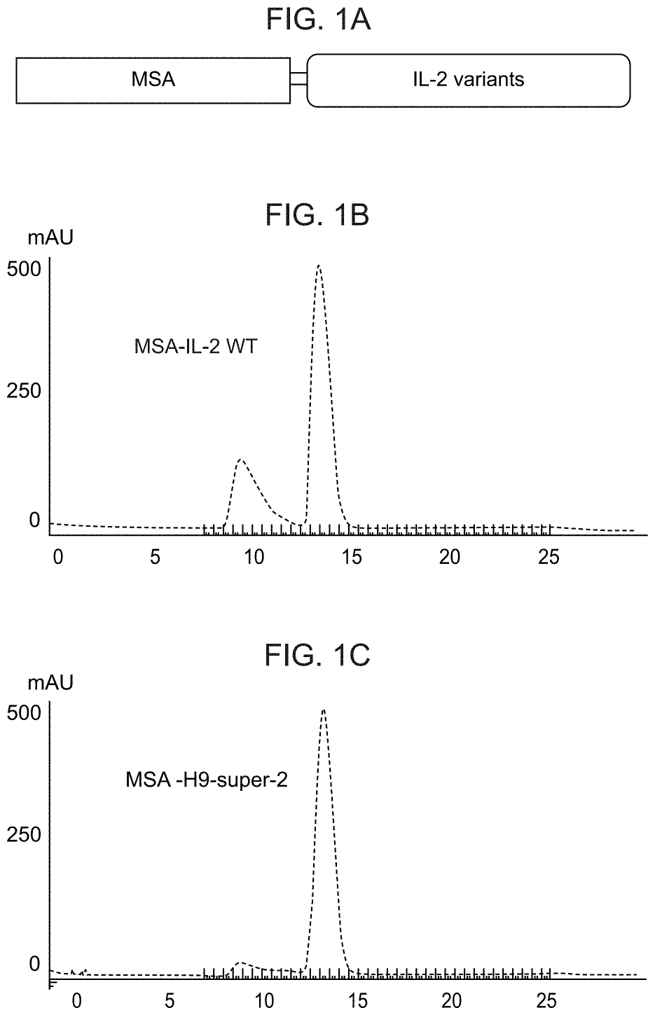

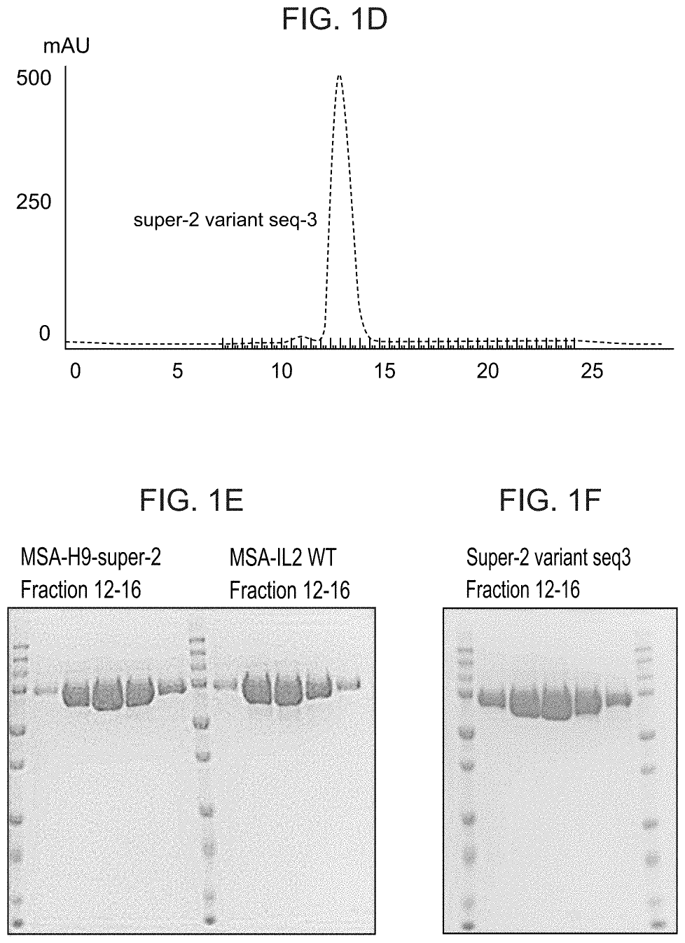

As shown in FIG. 1A, IL-2 variants were expressed as fusion proteins with mouse serum albumin (MSA) for half-life extension and recombinant expression from Expi-293 cells. FIG. 1B, 1C, and 1D show representative gel filtration traces (superdex 75) of MSA fusion proteins of human IL-2 WT (FIG. 1B), Super-2 (H9-super-2) (FIG. 1C), and in silico IL-2 variant SEQ ID NO:3 (variant seq-3) (FIG. 1D).

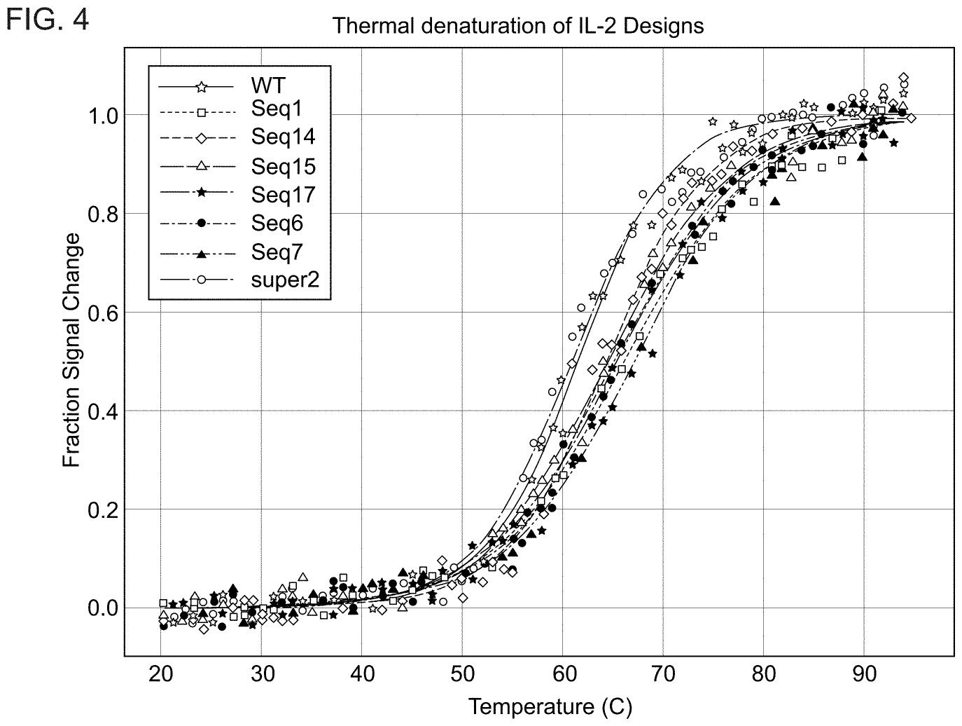

Normalized thermal melting curves of selected in silico designed variants monitored by circular dichroism is shown in FIG. 4. WT IL-2 and H9 Super-2 (super2) showed folding transitions at around 6° C., while the other selected in silico design variants are more thermally stable by about 5° C. Every in silico designed variant has improved thermostability compared to Wt. SEQ ID NO:6 is also included, although it seems to have the opposite effect in CD25± YT-1 cells than the rest of the clones.

### EXAMPLE 2: Stimulation of CD25⁻ and CD25⁺ Natural Killer (YT-1 cells)

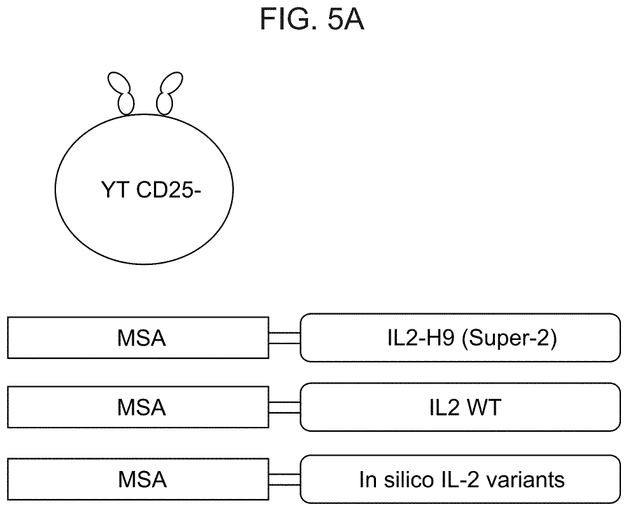

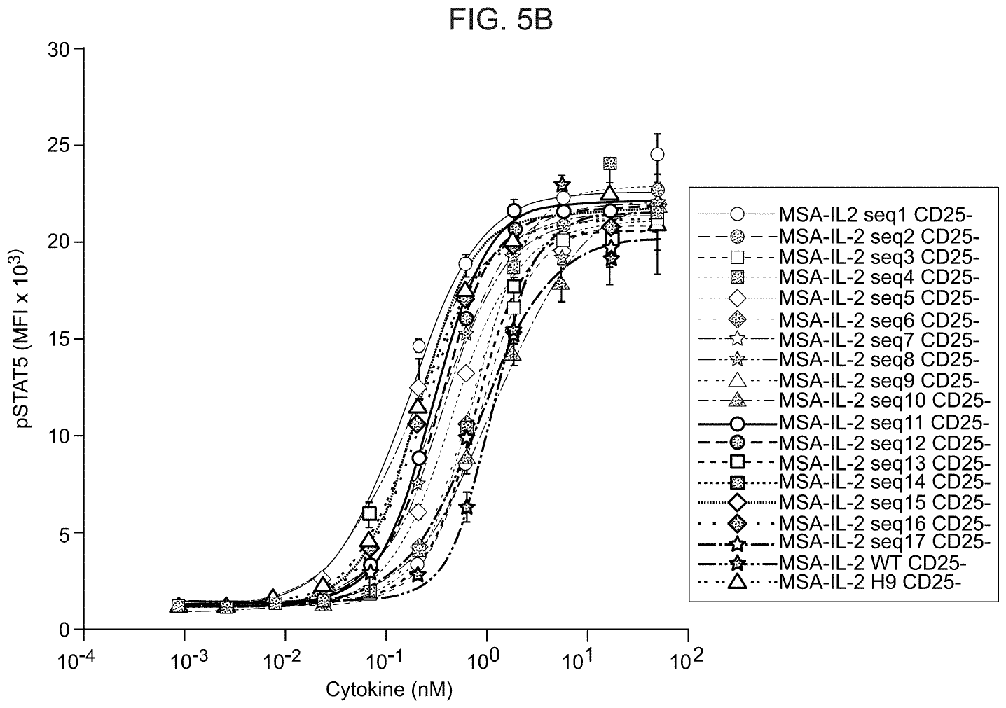

In silico designed IL-2 variants were tested for dose-response curves for STAT5 phosphorylation on CD25− YT-1 cells. Included for comparison is super-2 (H9) and WT IL-2. Most of the in silico designed variants exhibit left-shifted dose response curves indicating enhanced potency compared to WT IL-2, shown in FIG. 5B. 2×105 CD25+ or CD25− YT-1 cells were washed with FACS buffer and re-suspended in 200 μL FACS buffer with the indicated concentration of IL-2 variant per well in a 96-well plate. Cells were stimulated for 20 minutes at room temperature and then fixed by addition of formaldehyde to 1.5% and incubated for 10 min. Cells were permeabilized with 100% ice-cold methanol for 20 min on ice, followed by incubation at −80° C. overnight. Fixed, permeabilized cells were washed with excess FACS buffer and incubated with 50 μL Alexa647 conjugated anti-STAT5 pY694 (BD Biosciences, San Jose, Calif.) diluted 1:20 in FACS buffer for 20 minutes. Cells were washed twice in FACS buffer and mean cell fluorescence determined using the FL-4 channel of an Accuri C6 flow cytometer. Dose-response curves and EC50 values were calculated in GraphPad Prism after subtracting the mean cell fluorescence of unstimulated cells.

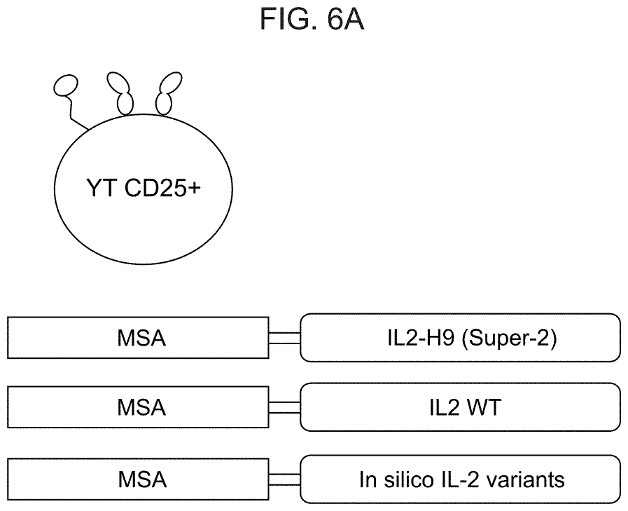

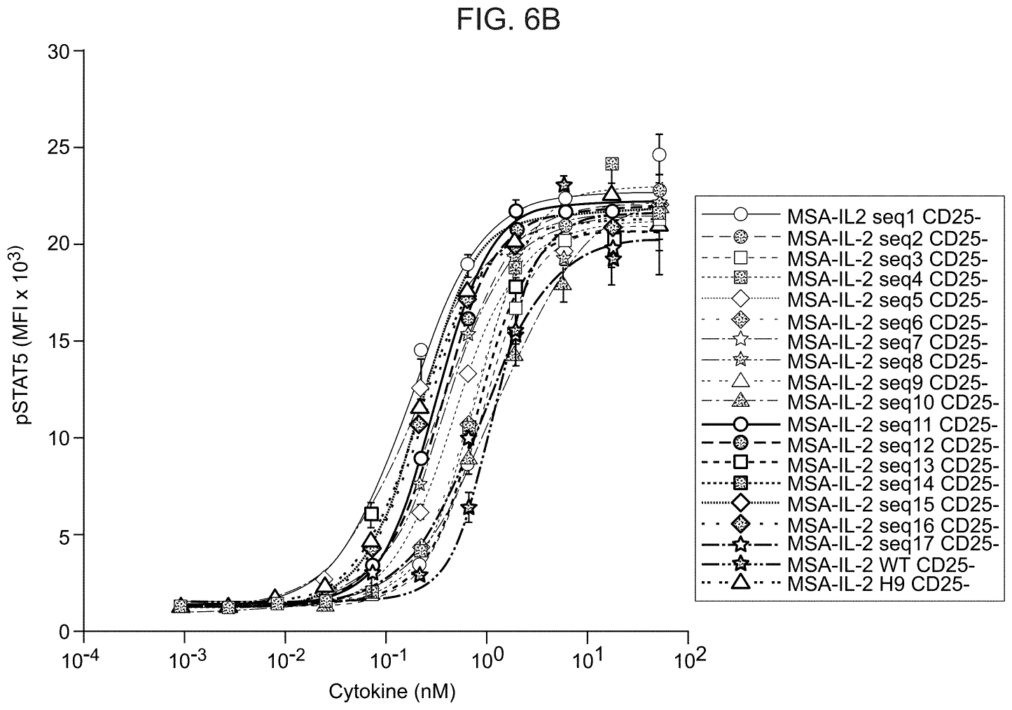

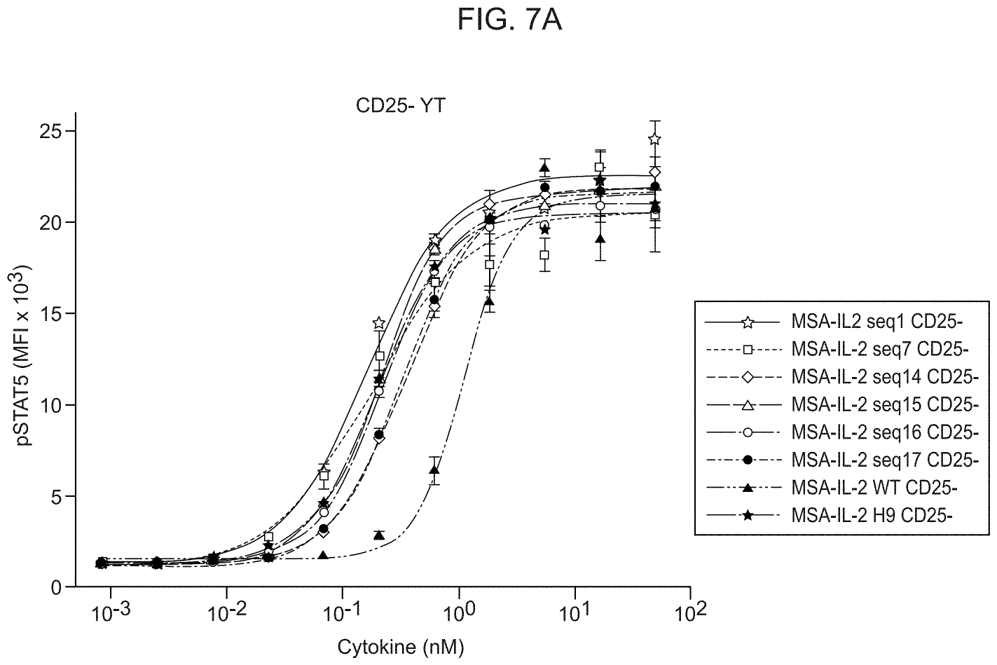

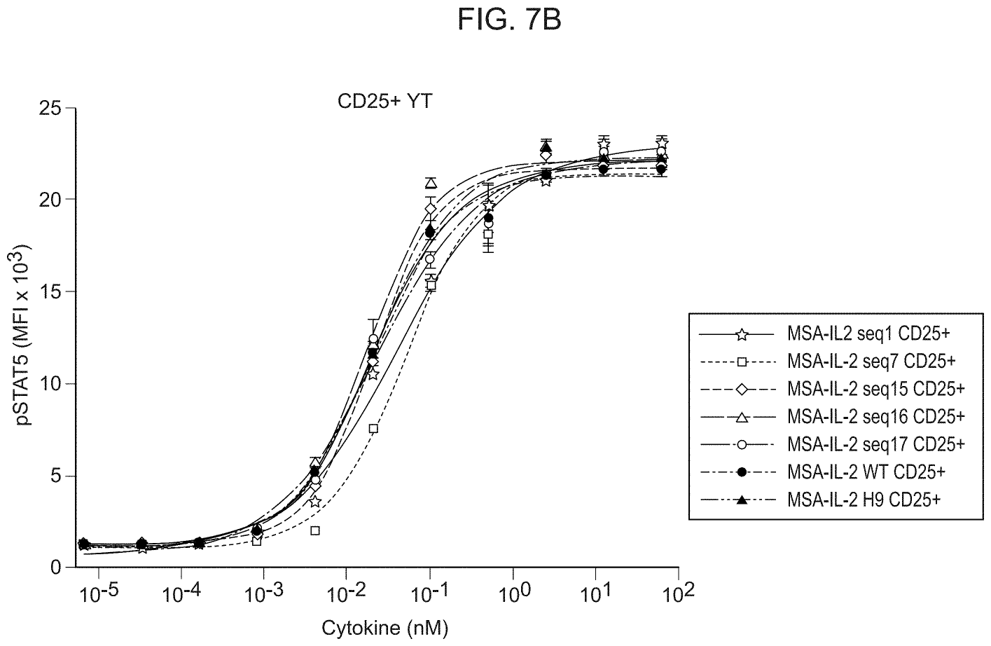

The most potent in silico designed IL-2 variants are shown for comparison tested for dose-response curves for STAT5 phosphorylation on CD25+ and CD25− YT-1 cells. Included for comparison is super-2 (H9) and wt IL-2, shown in FIG. 6B. Super-2 (also referred to as H9) is a IL-2 variant with mutations L80F; R81D; L85V; I86V; and I92F. The amino acid sequence of Super-2 is denoted by SEQ ID NO: 20.

SEQ ID NO: 20
(Super-2)
APTSSSTKKTQLQLEHLLLDLQMILNGINNYKNPKLTRMLTFKFYMPKKA
TELKHLQCLEEELKPLEEVLNLAQSKNFHFDPRDVVSNINVFVLELKGSE
TTFMCEYADETATIVEFLNRWITFCQSITSTLT

2×105 CD25+ or CD25− YT-1 cells were washed with FACS buffer and re-suspended in 200 μL FACS buffer with the indicated concentration of IL-2 variant per well in a 96-well plate. Cells were stimulated for 20 minutes at room temperature and then fixed by addition of formaldehyde to 1.5% and incubated for 10 min. Cells were permeabilized with 100% ice-cold methanol for 20 min on ice, followed by incubation at −80° C. overnight. Fixed, permeabilized cells were washed with excess FACS buffer and incubated with 50 μL Alexa647 conjugated anti-STAT5 pY694 (BD Biosciences, San Jose, Calif.) diluted 1:20 in FACS buffer for 20 minutes. Cells were washed twice in FACS buffer and mean cell fluorescence determined using the FL-4 channel of an Accuri C6 flow cytometer. Dose-response curves and EC50 values were calculated in GraphPad Prism after subtracting the mean cell fluorescence of unstimulated cells.

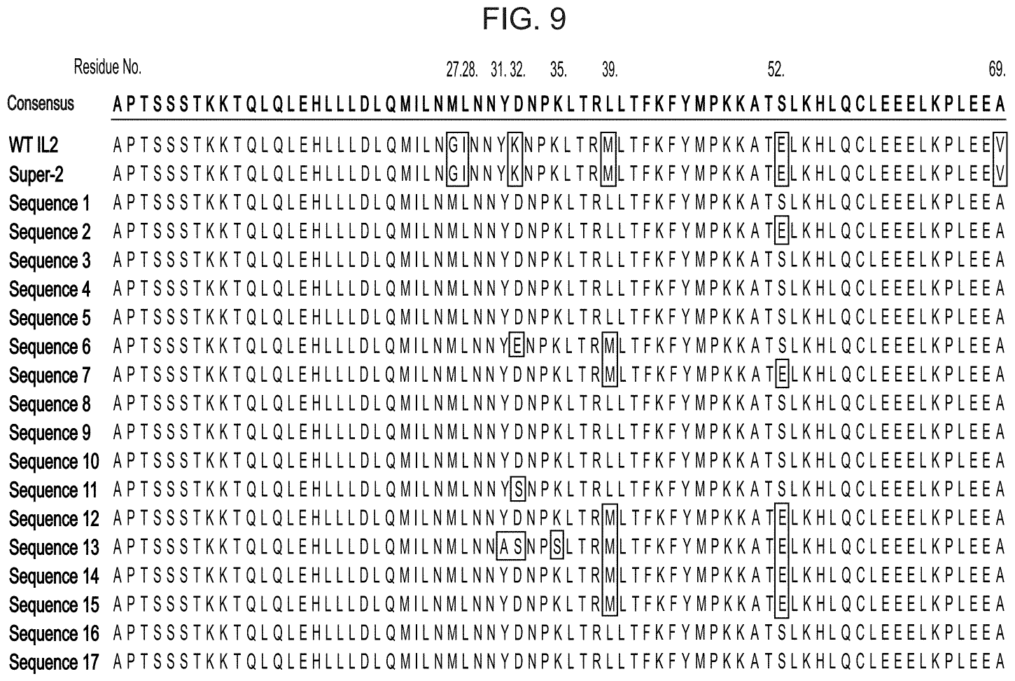

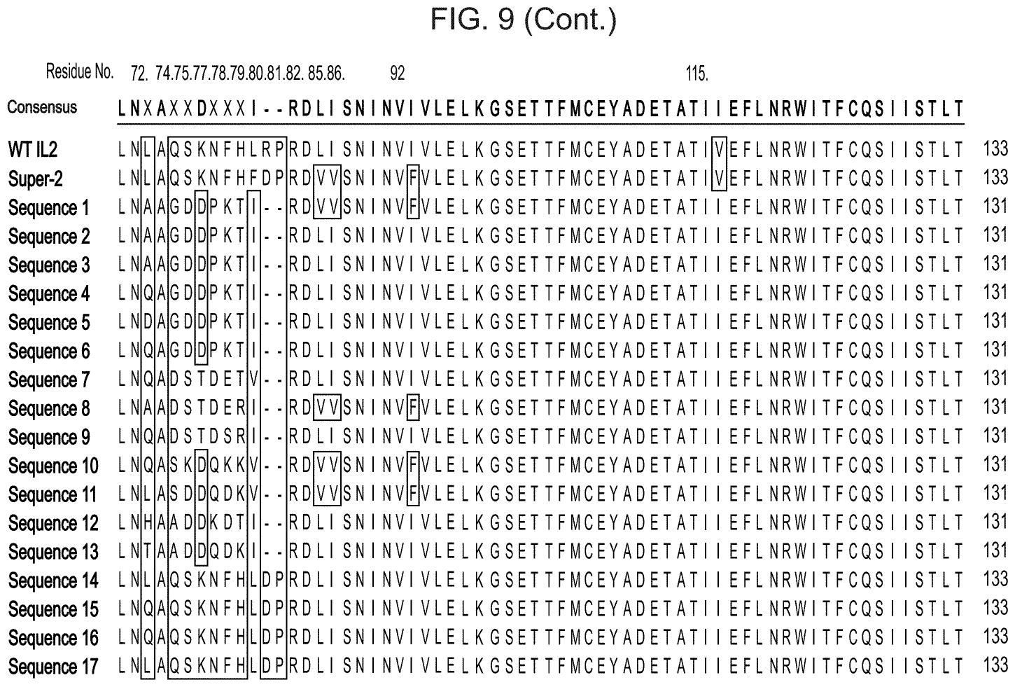

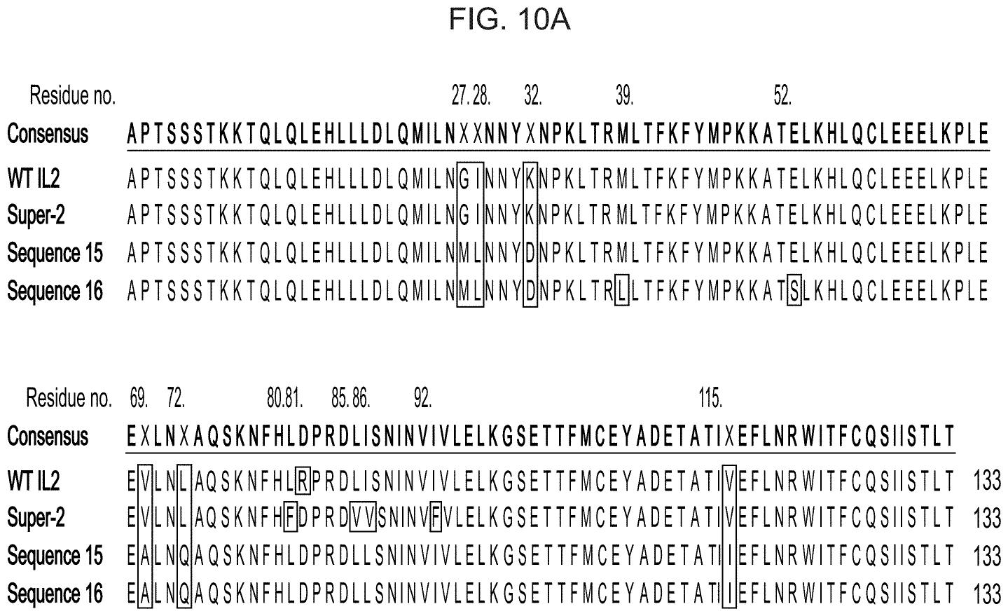

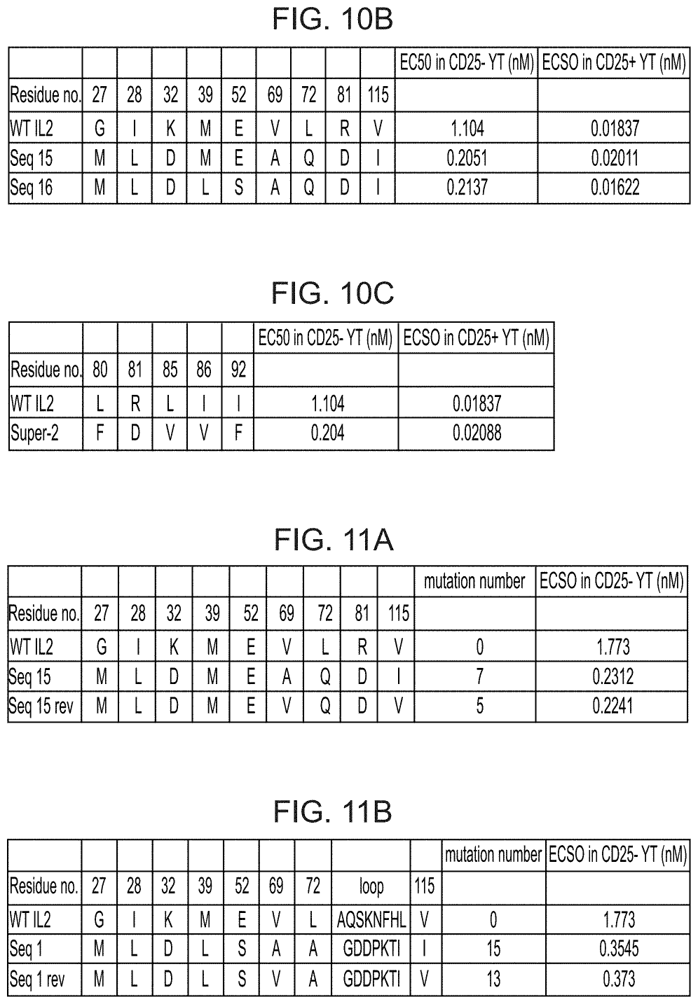

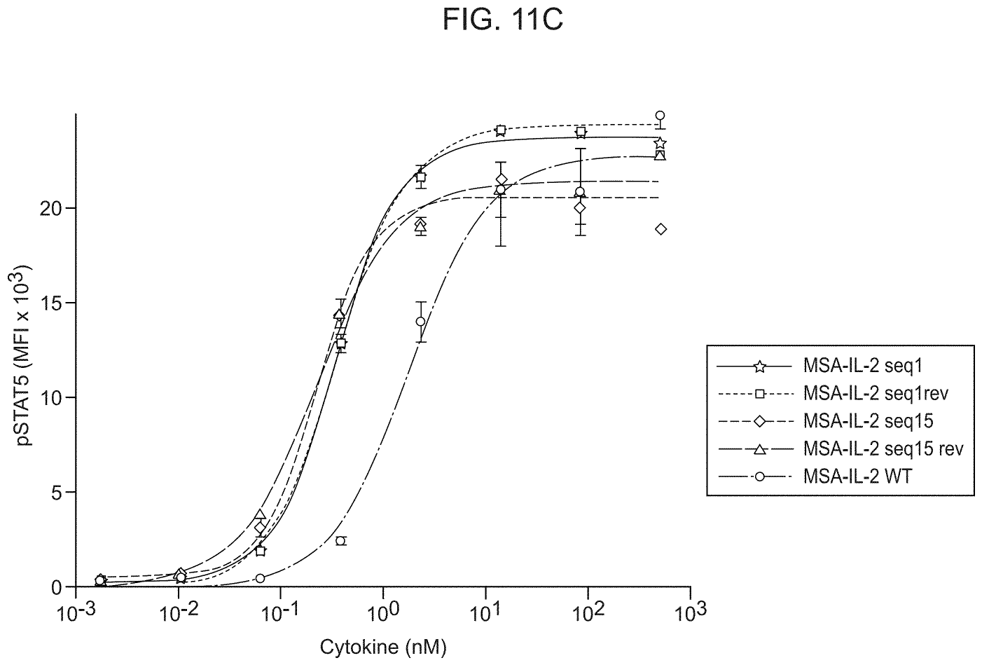

FIG. 8 is a table showing sequence differences between IL-2, super-2 and in silico IL-2 variants. EC50 values for STAT5 phosphorylation on CD25+ and CD25− YT-1 cells are shown in the right columns. FIG. 9 provides the sequences of all in silico designed IL-2 variants. FIG. 10A shows amino acid sequences of WT IL-2 (SEQ ID NO:19), Super-2 (SEQ ID NO: 20), and in silico designed IL-2 variants SEQ ID NO:15 (Sequence 15) and SEQ ID NO:16 (Sequence 16). FIG. 10B is a table showing sequence comparing the substitutions in IL-2 variants SEQ ID NO:15 (SEQ 15) and SEQ ID NO:16 (SEQ 16) compared to WT IL-2 (SEQ ID NO:19) and corresponding EC50 values for STAT5 phosphorylation in CD25− YT-1 cells and CD25+ YT-1 cells. FIG. 10C is a table showing sequence substitutions in Super-2 (SEQ ID NO: 20) compared to WT IL-2 and corresponding EC50 values for STAT5 phosphorylation on CD25− YT-+1 cells and CD25+ YT-1 cells.

### EXAMPLE 3: Binding Affinities of IL-2 Variants

TABLE 1
Binding affinity and EC₅₀ of IL-2 variants

| | | Kinetic fits | | | EC50 in | EC50 in |
|---|---|---|---|---|---|---|
| | SS | ka kd( s−1) KD (nM) | | | CD25⁻ | CD25⁺ |
| | KD | | Kd | KD | YT-1 | YT-1 |
| Construct | (nM) | Ka | (s−1) | (nM) | (nM) | (nM) |
| wt | 374 | 1.3e5 | 4.0e−3 | 30 | 1.104 | 0.01837 |
| H9 | 16.2 | 7.6e5 | 1.5e−3 | 2.1 | 0.204 | 0.02088 |
| sequence 1 | 61.3 | 2.7e5 | 6.6e−3 | 25 | 0.1606 | 0.03897 |
| sequence 2 | 411 | 1.7e5 | 5.5e−2 | 332 | 0.9324 | 0.04425 |
| sequence 3 | 366 | 1.5e5 | 4.2e−2 | 282 | 0.8217 | 0.03929 |
| sequence 4 | 389 | 1.5e5 | 5.0e−2 | 339 | 0.7419 | 0.1053 |
| sequence 5 | 362 | 1.6e5 | 4.7e−2 | 292 | 0.7559 | 0.1604 |
| sequence 6 | 398 | 1.5e5 | 4.6e−2 | 315 | 0.6747 | 0.009608 |
| sequence 7 | 85.4 | 4.6e5 | 2.6e−3 | 56.7 | 0.1706 | 0.05181 |
| sequence 8 | 14.2 | 7.7e5 | 4.4e−3 | 5.9 | 0.356 | 0.0327 |
| sequence 9 | 378 | 3.7e5 | 1.2e−1 | 330 | 0.7574 | 0.07403 |
| sequence 10 | 137 | 1.5e5 | 1.5e−2 | 105 | 1.168 | 0.0235 |
| sequence 11 | 22.7 | 4.1e5 | 3.6e−3 | 8.7 | 0.2908 | 0.01612 |
| sequence 12 | 67.6 | 4.6e5 | 2.3e−2 | 51.2 | 0.3209 | 0.03585 |
| sequence 13 | 207 | 2.1e5 | 3.6e−2 | 174 | 0.4382 | 0.03727 |
| sequence 14 | 196 | 4.2e5 | 7.2e−2 | 171 | 0.3527 | 0.01856 |
| sequence 15 | 498 | 2.8e5 | 6.4e−2 | 227 | 0.2051 | 0.02011 |
| sequence 16 | 290 | 3.2e5 | 7.1e−2 | 221 | 0.2137 | 0.01622 |
| sequence 17 | 268 | 3.8e5 | 9.1e−2 | 241 | 0.326 | 0.02188 |

Biacore measurements were performed on each of the variants for binding to IL-2Rβ. Many of the designed variants have a higher affinity for IL-2Rβ than WT IL-2, particularly sequences 1, 7 and 8. Sequence 6 has a lower affinity for IL-2Rβ, but is highly CD25 selective. Sequences 15, 16, and 17 have a binding affinity for IL-2Rβ similar to that of WT IL-2 but maintain a very potent EC50 for CD25− YT-1 cells. Variants with enhanced potency against CD25− YT-1 cells include for example, sequence 1, 7, 8, 11, 12, 13, 15, 16 and 17.

To perform the surface plasmon resonance (SPR) analysis, the test variant protein was made in Expi293 as described previously. IL-2Rβ was biotinylated and immobilized on the chip, and IL-2 variants were flowed over the chip. Experiments were performed on a BlAcore® T100 (GE Healthcare, Piscataway, N.J.).

Specific IL-2 variant residues were reverted back to wild-type amino acids at positions 69 and 115 (A69V and 1115V, respectively), with numbering relative to wild-type IL2 (SEQ ID NO:19).

### EXAMPLE 4: NK Cell Cytotoxicity Assay

The effect of in silico designed IL-2 variants on NK cell antibody-dependent cell-mediated cytotoxicity (ADCC) are assessed using an endothelial growth factor receptor (EGFR)-expressing squamous tumor cell line (SCC6) and an EGFR-specific monoclonal antibody.

Primary NK cells collected and isolated from healthy donors and assessed for cell purity. SCC6 target cells are labeled with 150 μCi 51Cr per 1×106 cells for 2 hours. Percent lysis is determined after 5 hours of co-culture of NK cells an various effector:target cell ratios with 51Cr-labelled SCC6 cells (e.g., 0:1. 1:1, 2:1, 5:1) in media alone or media containing EGFR-specific monoclonal antibody, IL-2, in silico designed IL-2 variants, or combinations thereof.

### EXAMPLE 5: IL-2 Variant effects on Memory T Cell and T-regulatory (T_{reg}) Cell Populations

The effect of in silico designed IL-2 variants on in vivo T cell populations can be assessed in C57B1/6 mice receiving IL-2, in silico designed IL-2 variants, or IL-2/anti-IL-2 monoclonal antibody complexes. Splenic cell suspensions are prepared from the spleens of treated mice and total cell counts of splenic CD3+, CD4+, and CD44 high memory T cells are assessed by flow cytometry using standard techniques familiar to persons of skill in the art.

### EXAMPLE 6: Assessing in vivo Toxicity of IL-2 variants

The in vivo toxicity of in silico designed IL-2 variants can be compared to WT IL-2 in an acute pulmonary edema model. C57B1/6 mice are intraperitoneally injected daily for 5 days with IL-2, in silico designed IL-2 variants, or IL-2/anti-IL-2 monoclonal antibody complexes. 6 days after the first treatment, lungs are removed and weighed before and after drying overnight at 58° C. under vacuum. Pulmonary wet weights are calculated by subtracting the initial pulmonary weight from the lung weights after dehydration.

### EXAMPLE 7: In vivo Anti-Tumor Activity of IL-2 Variants

The anti-tumor activity of IL-2 variants can be tested in an in vivo mouse model. B16F10 melanoma cells are injected into the upper dermis of the back of C57B1/6 mice. After tumor nodules attain a size of approximately 15 mm2, mice are injected daily for 5 days with IL-2, in silico designed IL-2 variants, or IL-2/anti-IL-2 monoclonal antibody complexes. Following treatment, serum and tumor lysates are prepared and analyzed for standard tumor markers.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. For example, although IL-2 is referred to throughout the specification, one of skill in the art would appreciate that the methods and compositions described herein are equally applicable to other cytokines, for example, granulocyte-macrophage colony-stimulating factor (GM-CSF), IL-2, IL-3, IL-5, IL-6, or IL-15 with this property. Thus, the invention also includes variants of GM-CSF, IL-2, IL-3, IL-5, IL-6, and IL-15 with increased binding affinity for their respective receptors, as compared to wild-type, and methods for identifying and using those variants.

1. A variant human IL-2 protein comprising one or more amino acid changes, wherein the one or more amino acid changes is selected from the group consisting of:
(i) an amino acid substitution to methionine at position 27 (G27M);
(ii) an amino acid substitution at position 28;
(iii) an amino acid substitution to alanine at position 31 (Y31A);
(iv) an amino acid substitution at position 32 selected from the group consisting of a substitution to aspartic acid (K32D), a substitution to glutamic acid (K32E), and a substitution to serine (K32S);
(v) an amino acid substitution to serine at position 35 (K35S);
(vi) an amino acid deletion at position 82 (P82Δ); and
(vii) an amino acid substitution to isoleucine at position 115 (V115I), relative to the amino acid sequence of wild-type human IL-2 protein represented by SEQ ID NO:19.

2. The variant human IL-2 protein of claim 1, wherein the variant human IL-2 protein comprises an amino acid substitution at position 28, relative to the amino acid sequence of wild-type IL-2 protein represented by SEQ ID NO:19.

3. The variant human IL-2 protein of claim 2, further comprising an amino acid substitution at position 27, relative to the amino acid sequence of wild-type IL-2 protein represented by SEQ ID NO:19.

4. The variant human IL-2 protein of claim 3, wherein the amino acid substitution at position 27 is a substitution to methionine (G27M).

5. The variant human IL-2 protein of claim 4, further comprising an amino acid substitution at position 32, relative to the amino acid sequence of wild-type IL-2 protein represented by SEQ ID NO:19.

6. The variant human IL-2 protein of claim 5, wherein the amino acid substitution at position 32 is selected from the group consisting of: a substitution to aspartic acid (K32D), a substitution to glutamic acid (K32E), and a substitution to serine (K32S).

7. The variant human IL-2 protein of claim 6, wherein the amino acid substitution at position 32 is a substitution to aspartic acid (K32D).

8. The variant human IL-2 protein of claim 7, wherein the amino acid substitution at position 28 comprises a substitution to leucine (I28L).

9. The variant human IL-2 protein of claim 8, further comprising an amino acid substitution at position 72.

10. The variant human IL-2 protein of claim 9, wherein the amino acid substitution at position 72 is selected from the group consisting of: a substitution to alanine (L72A), a substitution to glutamine (L72Q), a substitution to aspartic acid (L72D), a substitution to histidine (L72H), and a substitution to threonine (L72T).

11. The variant human IL-2 protein of claim 10, wherein the amino acid substitution at position 72 comprises a substitution to glutamine (L72Q).

12. The variant human IL-2 protein of claim 11, further comprising an amino acid substitution at position 81.

13. The variant human IL-2 protein of claim 12, wherein the amino acid substitution at position 81 comprises a substitution to aspartic acid (R81 D).

14. The variant human IL-2 protein of claim 13, wherein the amino acid changes comprise or consist of an amino acid substitution to methionine at position 27 (G27M), an amino acid substitution to leucine at position 28 (I28L), an amino acid substitution to aspartic acid at position 32 (K32D), an amino acid substitution to glutamine at position 72 (L72Q), and an amino acid substitution to aspartic acid at position 81 (R81 D).

15. The variant human IL-2 protein of claim 10, wherein the amino acid changes comprise or consist of an amino acid substitution to methionine at position 27 (G27M), an amino acid substitution to leucine at position 27 (I28L), an amino acid substitution to aspartic acid at position 32 (K32D), an amino acid substitution to leucine at position 39 (M39L), an amino acid substitution to serine at position 52 (E52S), an amino acid substitution to alanine at position 72 (L72A), a substitution of the region encompassing amino acid residues 74-80 with GDDPKTI, an amino acid deletion at position 81 (R81Δ), an amino acid deletion at position 82 (P82Δ), an amino acid substitution to valine at position 85 (L85V), an amino acid substitution to valine at position 86 (I86V), and an amino acid substitution to phenylalanine at position 92 (I92F).

16. The variant human IL-2 protein of claim 1, wherein the variant human IL-2 protein has amino acid changes of no more than twenty amino acid residues relative to the amino acid sequence of wild-type human IL-2 protein represented by SEQ ID NO: 19.

17. The variant human IL-2 protein of claim 1, wherein the variant human IL-2 protein has a greater potency for activation of IL-2 signaling pathways in cells lacking CD25 expression relative to wild-type IL-2.

18. The variant human IL-2 protein of claim 1, wherein the variant human IL-2 protein has an EC50 for activation of IL-2 signaling pathways in cells lacking CD25 expression at least two-fold lower than wild-type IL-2.

19. The variant human IL-2 protein of claim 1, wherein the variant human IL-2 protein has increased binding affinity for human CD122, as measured in a surface plasmon resonance binding assay.

20. The variant human IL-2 protein of claim 1, wherein the variant human IL-2 protein has a greater potency for activation of IL-2 signaling pathways in cells lacking CD25 expression, but does not have a substantially increased binding affinity for CD122 relative to wild type IL-2.

21. The variant human IL-2 protein of claim 1, wherein the variant human IL-2 protein is fused to a human IgG Fc domain.

22. The variant human IL-2 protein of claim 21, wherein the human IgG Fc domain is an IgG1, IgG2, IgG3, or IgG4 Fc domain.

23. A nucleic acid encoding a variant human IL-2 protein according to claim 1.

24. A vector comprising a nucleic acid encoding a variant human IL-2 protein according to claim 23.

25. A recombinant cell comprising a nucleic acid according to claim 23.

26. A pharmaceutical composition comprising a variant human IL-2 protein according to claim 1, and a pharmaceutically acceptable carrier.

27. A method of treating a disease in a subject in need thereof, the method comprising administering to the subject the pharmaceutical composition of claim 26.

28. A method of treating a disease in a subject in need of expansion of T cells, the method comprising administering an effective amount of the pharmaceutical composition of claim 26.

29. The method of claim 27, wherein the disease is a hyperproliferative disease.

30. The method of claim 29, wherein the disease is cancer.

31. A method for in silico design of a cytokine based on crystallographic topology of the ligand receptor interaction, the method comprising: identifying locations where packing can be improved in order to increase binding; redesigning core positions and testing in a computer model to identify positions where improvements can be introduced: generating backbone structure ensembles while allowing the entire core to redesign while monitoring the residue changes in the identified regions; visually selecting a subset of resides that improve packing; holding identities of the subset and iteratively performing the redesign until the sequences converge; generating a coding sequence for the in silico designed cytokine.


SEQUENCE LISTING (20 sequences)

SEQ ID NO: 1 - protein, 131 aa, Homo Sapiens
APTSSSTKKTQLQLEHLLLDLQMILNMLNNYDNPKLTRLLTFKFYMPKKATSLKHLQCLEEELKPLEEALNAAGDDPKTIRDVVSNINVFVLELKGSETTFMCEYADETATIIEFLNRWITFCQSIISTLT

SEQ ID NO: 2 - protein, 131 aa, Homo Sapiens
APTSSSTKKTQLQLEHLLLDLQMILNMLNNYDNPKLTRLLTFKFYMPKKATELKHLQCLEEELKPLEEALNAAGDDPKTIRDLISNINVIVLELKGSETTFMCEYADETATIIEFLNRWITFCQSIISTLT

SEQ ID NO: 3 - protein, 131 aa, Homo Sapiens
APTSSSTKKTQLQLEHLLLDLQMILNMLNNYDNPKLTRLLTFKFYMPKKATSLKHLQCLEEELKPLEEALNAAGDDPKTIRDLISNINVIVLELKGSETTFMCEYADETATIIEFLNRWITFCQSIISTLT

SEQ ID NO: 4 - protein, 131 aa, Homo Sapiens
APTSSSTKKTQLQLEHLLLDLQMILNMLNNYDNPKLTRLLTFKFYMPKKATSLKHLQCLEEELKPLEEALNQAGDDPKTIRDLISNINVIVLELKGSETTFMCEYADETATIIEFLNRWITFCQSIISTLT

SEQ ID NO: 5 - protein, 131 aa, Homo Sapiens
APTSSSTKKTQLQLEHLLLDLQMILNMLNNYDNPKLTRLLTFKFYMPKKATSLKHLQCLEEELKPLEEALNDAGDDPKTIRDLISNINVIVLELKGSETTFMCEYADETATIIEFLNRWITFCQSIISTLT

SEQ ID NO: 6 - protein, 131 aa, Homo Sapiens
APTSSSTKKTQLQLEHLLLDLQMILNMLNNYENPKLTRMLTFKFYMPKKATSLKHLQCLEEELKPLEEALNQAGDDPKTIRDLISNINVIVLELKGSETTFMCEYADETATIIEFLNRWITFCQSIISTLT

SEQ ID NO: 7 - protein, 131 aa, Homo Sapiens
APTSSSTKKTQLQLEHLLLDLQMILNMLNNYDNPKLTRMLTFKFYMPKKATELKHLQCLEEELKPLEEALNQADSTDETVRDLISNINVIVLELKGSETTFMCEYADETATIIEFLNRWITFCQSIISTLT

SEQ ID NO: 8 - protein, 131 aa, Homo Sapiens
APTSSSTKKTQLQLEHLLLDLQMILNMLNNYDNPKLTRLLTFKFYMPKKATSLKHLQCLEEELKPLEEALNAADSTDERIRDVVSNINVFVLELKGSETTFMCEYADETATIIEFLNRWITFCQSIISTLT

SEQ ID NO: 9 - protein, 131 aa, Homo Sapiens
APTSSSTKKTQLQLEHLLLDLQMILNMLNNYDNPKLTRLLTFKFYMPKKATSLKHLQCLEEELKPLEEALNQADSTDSRIRDLISNINVIVLELKGSETTFMCEYADETATIIEFLNRWITFCQSIISTLT

SEQ ID NO: 10 - protein, 131 aa, Homo Sapiens
APTSSSTKKTQLQLEHLLLDLQMILNMLNNYDNPKLTRLLTFKFYMPKKATSLKHLQCLEEELKPLEEALNQASKDQKKVRDVVSNINVFVLELKGSETTFMCEYADETATIIEFLNRWITFCQSIISTLT

SEQ ID NO: 11 - protein, 131 aa, Homo Sapiens
APTSSSTKKTQLQLEHLLLDLQMILNMLNNYSNPKLTRLLTFKFYMPKKATSLKHLQCLEEELKPLEEALNLASDDQDKVRDVVSNINVFVLELKGSETTFMCEYADETATIIEFLNRWITFCQSIISTLT

SEQ ID NO: 12 - protein, 131 aa, Homo Sapiens
APTSSSTKKTQLQLEHLLLDLQMILNMLNNYDNPKLTRMLTFKFYMPKKATELKHLQCLEEELKPLEEALNHAADDKDTIRDLISNINVIVLELKGSETTFMCEYADETATIIEFLNRWITFCQSIISTLT

SEQ ID NO: 13 - protein, 131 aa, Homo Sapiens
APTSSSTKKTQLQLEHLLLDLQMILNMLNNASNPSLTRMLTFKFYMPKKATELKHLQCLEEELKPLEEALNTAADDQDKIRDLISNINVIVLELKGSETTFMCEYADETATIIEFLNRWITFCQSIISTLT

SEQ ID NO: 14 - protein, 133 aa, Homo Sapiens
APTSSSTKKTQLQLEHLLLDLQMILNMLNNYDNPKLTRMLTFKFYMPKKATELKHLQCLEEELKPLEEALNLAQSKNFHLDPRDLISNINVIVLELKGSETTFMCEYADETATIIEFLNRWITFCQSIISTLT

SEQ ID NO: 15 - protein, 133 aa, Homo Sapiens
APTSSSTKKTQLQLEHLLLDLQMILNMLNNYDNPKLTRMLTFKFYMPKKATELKHLQCLEEELKPLEEALNQAQSKNFHLDPRDLISNINVIVLELKGSETTFMCEYADETATIIEFLNRWITFCQSIISTLT

SEQ ID NO: 16 - protein, 133 aa, Homo Sapiens
APTSSSTKKTQLQLEHLLLDLQMILNMLNNYDNPKLTRLLTFKFYMPKKATSLKHLQCLEEELKPLEEALNQAQSKNFHLDPRDLISNINVIVLELKGSETTFMCEYADETATIIEFLNRWITFCQSIISTLT

SEQ ID NO: 17 - protein, 133 aa, Homo Sapiens
APTSSSTKKTQLQLEHLLLDLQMILNMLNNYDNPKLTRLLTFKFYMPKKATSLKHLQCLEEELKPLEEALNLAQSKNFHLDPRDLISNINVIVLELKGSETTFMCEYADETATIIEFLNRWITFCQSIISTLT

SEQ ID NO: 18 - protein, 153 aa, Homo Sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 19 - protein, 133 aa, Homo Sapiens [sequence omitted]

SEQ ID NO: 20 - protein, 133 aa, Homo Sapiens
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT